(19) United States
(12) Patent Application Publication
(10) Pub. No.: US 2007/0282422 A1
(43) Pub. Date: Dec. 6, 2007

(54) Medical devices and methods for local delivery of elastin-stabilizing compounds

(75) Inventors: David Paul Biggs, Bloomington, IN (US); Anthony O. Ragheb, West Lafayette, IN (US); Patrick H. Ruane, Redwood City, CA (US)

Correspondence Address: BRINKS HOFER GILSON & LIONE/CHICAGO/COOK, PO BOX 10395, CHICAGO, IL, 60610, US

(73) Assignees: Cook Incorporated; MED Institute, Inc.

(21) Appl. No.: 11/800,217

(22) Filed: May 4, 2007

Related U.S. Application Data
(60) Provisional application No. 60/799,608, filed on May 10, 2006.

(51) Int. Cl. A61F 2/82; A61F 2/02

(52) U.S. Cl. 623/1.13; 424/423; 623/1.42

(57) ABSTRACT

This invention relates to medical devices and an elastin-stabilizing compound, such as a phenolic tannin, the medical device being adapted to release the elastin-stabilizing compound within a body of a patient. This invention also relates to medical devices and methods of treatment of disease, such as aneurysms and aortic dissection. Medical devices may include coated stents, grafts, stent grafts, balloons and catheters.

### RELATED APPLICATIONS

The present patent document claims the benefit of the filing date under 35 U.S.C. §119(e) of Provisional U.S. Patent Application Ser. No. 60/799,608, filed May 10, 2006, which is hereby incorporated by reference.

### TECHNICAL FIELD

This invention relates generally to human and veterinary medical devices and, more particularly, to implantable medical devices incorporating elastin-stabilizing compounds. The invention also relates to methods of treatment and kits and to treating an aorta wall adjacent to an aortic aneurysm as a preventative measure.

### BACKGROUND

Diseases of aorta are common in the general population and may include endovascular disease, including aneurysms and aortic dissections.

Endovascular disease may be characterized by weakened vessels due to elastin breakdown, which results in dilation of vessels and aneurysm. An aneurysm is a sac formed by localized dilatation of the wall of an artery, a vein, or the heart. Common areas where aneurysms occur and cause adverse medical conditions include the coronary arteries, the carotid arteries, various cerebral arteries, and the thoracic and abdominal aorta as well as iliac and femoral arteries. When a local dilatation of a vessel occurs, irregular blood flow patterns result in the lumen of the vessel, sometimes leading to clot formation. Typically, the wall of the vessel also progressively dilates and weakens, often resulting in vessel rupture. Vessel rupture, in turn, often causes dramatic negative consequences such as a stroke, when a cerebral vessel ruptures, or even death, when an abdominal aortic aneurysm (“AAA”) ruptures. Continued degeneration can result in an increase in aneurysm size due to thinning of the medial connective tissue of the aorta and loss of elastin.

Aortic dissections occur when the inner layer of the aorta's artery wall splits open (dissects). The normal aorta contains collagen, elastin, and smooth muscle cells that contribute to the intima, media, and adventitia, which are the layers of the aorta. Hypertension with aging is believed to contribute to degenerative changes that may lead to breakdown of the collagen, elastin, and smooth muscle cells and ultimately dissection of the aorta. Aortic dissection is more likely to occur where pressure on the artery wall from blood flow is high, such as the proximal aorta or the ascending aorta (the first segment of the aorta). When the aortic wall splits, the pulses of blood can penetrate the artery wall and its inner layer, causing the aorta to tear or split further. This tear usually continues distally (away from the heart) down the descending aorta and into its major branches. Less often, the tear may run proximally (back toward the heart). Aortic dissection can also start in the descending (distal) segment of the aorta.

In light of these consequences, improved devices and methods of treating and/or preventing aneurysms and aortic dissections are constantly being sought. Although the following discussion focuses on AAA treatment and prevention, it is equally applicable to endovascular disease in other locations, and aortic dissections.

Various implantable medical devices are advantageously inserted within various portions of the body. Minimally invasive techniques and instruments for placement of intralumenal medical devices have been developed to treat and repair undesirable conditions within body vessels including treatment of conditions that affect blood flow such as abdominal aortic aneurysm. Various percutaneous methods of implanting medical devices within the body using intralumenal transcatheter delivery systems can be used to treat a variety of such conditions. One or more intralumenal medical devices, such as tubular stent grafts, can be introduced to a point of treatment within a body vessel using a delivery catheter device passed through the vasculature communicating between a remote introductory location and the implantation site, and released from the delivery catheter device at the point of treatment within the body vessel. Intralumenal medical devices can be deployed in a body vessel at a point of treatment and the delivery device subsequently withdrawn from the vessel, while the medical device is retained within the vessel to provide sustained improvement in valve function or to increase vessel patency. For example, an implanted stent graft can improve vessel function by permitting relatively less turbulent fluid flow through the stent graft conduit bridging the site of an aneurysm.

### SUMMARY

In one embodiment, the invention provides a medical device and an elastin-stabilizing compound, the medical device being adapted to release an elastin-stabilizing compound within a body of a patient. The medical device may be implantable. The elastin-stabilizing compound may be a phenolic tannin. The phenolic tannin may be a hydrolyzable tannin or a condensed tannin. The phenolic tannin may be selected from gallotannins, ellagitannins, epicatechins, catechins, proanthocyanidins, derivatives, and mixtures thereof. The phenolic tannin may be selected from the group of compounds: tannic acid; β-1,2,3,4,6-Pentagalloyl-O-D-Glucopyranose; β-1,2,2,3,6-Pentagalloyl-O-D-glucose; 1,2,3,6-tetragalloyl glucose; 1,3,4,6-tetragalloyl glucose; Aceritannin=2,6-di-O-galloyl-1,5-anhydro-D-glucitol; Hamamelitannin; Ellagitannin; Eugeniin; Casuarictin; Corilagin; Geraniin; Davidiin; Castalagin; Vescalagin; Euphorbin; Oenethein B; Epicatechenin; Catechin; Epigallocatechin; Gallocatechin; Epiafzelechin; Afzelechin; Epicatechin (4β->8)-catechin; Epicatechin (4β->8)-epicatechin; Catechin (4α->8)-catechin; Catechin (4α->8)-epicatechin; Sorghum procyanidin; epicatechin-[(4b->8)-epicatechin]15-(4b->8)-catechin; Aurantinidin; Cyanidin; Delphinidin; Europinidin; Luteolinidin; Pelargonidin; Malvidin; Peonidin; Petunidin; Apigeninidin; Robinetinidin; Fisetinidin; Guibourtinidin; Robinetinidol-(4α->8)-catechin-(6α->4a)-robinetinidol; Profisetinidin; Epicatechin (2β-->7,4β-->8)-epicatechin; Leucofisetinidin; Leucopelargonidin; Leucocyanidin; Leucodelphinidin; Leucoapigeninidin; Leucoluteolinidin, derivatives, and mixtures thereof. The phenolic tannin may be pentagalloyl glucose (PGG). The medical device may be an implantable medical device. The implantable medical device may be an endolumenal medical device such as a stent, the elastin-stabilizing compound releasably associated with the stent. The stent may comprise a plurality of interconnected struts and bends, the elastin-stabilizing compound releasably associated with the struts, bends, or a combination thereof. Alternatively, the stent may comprise a plurality of Z-STENTS®. The implantable medical device may be a stent graft comprising a support frame attached to a flexible tubular covering, the elastin-stabilizing compound releasably associated with at least a portion of the stent graft. The medical device may also include at least one surface adapted for contact with a body vessel wall and comprising the elastin-stabilizing compound coated on at least a portion of the at least one surface. The medical device may include an elongated member having a lumen extending longitudinally along the length of the elongated member, the elongated member having an ablumenal surface and a lumenal surface, where the elastin-stabilizing compound may be releasably associated with at least a portion of at least one surface of the elongated member. For example, the implantable medical device may be configured as a stent graft having an elongated member configured as a flexible tubular covering forming at least a portion of the ablumenal surface that also includes a radially expandable support frame comprising a plurality of hoops attached to the elongated member. The lumen may form a fluid conduit defined by the lumenal surface. The elastin-stabilizing compound may be releasably associated with at least a portion of the ablumenal surface of the elongated member. The implantable medical device may also be a coated stent comprising a plurality of interconnected struts and bends, with a coating comprising the elastin-stabilizing compound releasably associated with at least one strut, bend, or a combination thereof. The coating may comprise one or more layers containing the elastin-stabilizing compound and a bioabsorbable polymer. The layers may include varying amounts of the elastin-stabilizing compound(s). The implantable medical device may also be a graft comprising an elastin-stabilizing compound. The elastin-stabilizing compound may be contained within a reservoir, a well or a groove. Alternatively, the elastin-stabilizing compound may be in or disposed on a separate carrier layer.

In another embodiment, the invention provides a method of treating an aneurysm or an aortic dissection including providing the medical device of this invention.

In yet another embodiment, the invention provides a method for preventing an aortic dissection including providing the medical device of this invention.

In yet another embodiment, the invention provides a method of treating an aneurysm or an aortic dissection comprising radially expanding a medical device in a lumen with a balloon catheter, wherein the balloon catheter releases an elastin-stabilizing compound.

In a further embodiment, the invention provides a kit including a medical device and a balloon catheter comprising elastin-stabilizing compound.

### BRIEF DESCRIPTION OF THE DRAWINGS

FIG. 1 is a schematic showing the Phenylpropanoic or Shikimic acid pathways for the synthesis of phenolic compounds.

FIG. 2A is a side view of a coated expandable vascular stent endolumenal medical device.

FIG. 2B is a cross section of a strut of an implantable medical device comprising a single-layer coating configuration.

FIG. 2C is a cross section of a strut of an implantable medical device comprising a two-layer coating configuration.

FIG. 2D is a cross section of a strut of an implantable medical device comprising an alternate two-layer coating configuration.

FIG. 2E is a cross section of a strut of an implantable medical device comprising another alternate two-layer coating configuration.

FIG. 3A is a side view of a first stent graft implantable medical device.

FIG. 3B is a side view of a second stent graft implantable medical device.

FIG. 4 is a perspective view of a third stent graft implantable medical device comprising a two-layer graft material.

FIG. 5A is a partial, enlarged top view of a portion of a medical device.

FIGS. 5B-5D are enlarged cross-sectional views along lines B-B′ of the medical device of FIG. 5A.

FIG. 6 is a medical device configured as a coated balloon.

FIG. 7 is a medical device configured as a flexible material in an annular configuration.

FIG. 8 is a radial cross section of an exemplary medical device.

### DETAILED DESCRIPTION

The present disclosure describes medical devices, which comprise elastin-stabilizing compounds (such as, phenolic tannins), and methods of using these medical devices to stop or prevent breakdown of host connective tissue and treat variety of diseases and conditions, including endovascular disease including aneurysms and aortic dissections. The medical device can be configured to provide a disease treatment by providing an effective amount of a phenolic tannin compound proximate to a disease site within a body vessel. For example, the medical device can release or retain a phenolic tannin at a desired rate within a blood vessel upon placement proximate to an aneurysm or aortic dissection. By providing phenolic tannins with the device, the progression of local endovascular disease or aortic dissection may be mitigated, stopped and/or reversed, preventing further weakening and dilation of the vessel wall or splitting of the layers of aorta. These types of devices may be used for treatment of aneurysms, especially aortic abdominal aneurysms and for treatment or prevention of aortic dissections.

Researchers have hypothesized that the development, expansion and rupture of AAAs and aortic dissections are related to connective tissue destruction. For a discussion of this hypothesis, see for example, “Pharmacologic suppression of experimental abdominal aortic aneurysms: A comparison of doxycycline and four chemically modified tetracyclines,” Curci, John A., Petrinec, Drazen, et al., Journal of Vascular Surgery, December 1998, vol. 28, no. 6, 1082-1093. Connective tissue destruction, in turn, has been linked to the presence of a number of enzymes which break down components of blood vessel wall connective tissues, such as elastin. Examples of such “elastolytic” enzymes include serine proteinases and metalloproteinases (MMPs), which are derived from activated vascular cells and infiltrating inflammatory cells. It has been found that increased levels of some elastolytic enzymes are typically present in AAAs. Elastin degeneration may be stopped or prevented by locally delivering pharmacological agents capable of stabilizing elastin. The present invention uses the unique properties of phenolic compounds, and specifically phenolic tannin compounds, to develop a method of stabilizing elastin. Stabilizing elastin may be desirable to stop and/or prevent further progression and/or development of a vascular disease, such aneurysm, and especially AAA; and aortic dissections.

### Definitions

The following detailed description and appended drawings describe and illustrate various exemplary embodiments of the invention. The description and drawings serve to enable one skilled in the art to make and use the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

The recitation of “about” or “substantially” used with reference to a quantity, such as an angle, includes variations in the recited quantity that are equivalent to the quantity recited, for instance an amount that is insubstantially different from a recited quantity for an intended purpose or function.

As used herein, the term “implantable” refers to an ability of a medical device to be positioned at a location within a body for any suitable period of time, such as within a body vessel. Furthermore, the terms “implantation” and “implanted” refer to the positioning of a medical device at a location within a body, such as within a body vessel. Implantable medical devices can be configured for transient placement within a body vessel during a medical intervention (e.g., seconds, minutes, hours), or to remain in a body vessel for a prolonged period of time after an implantation procedure (e.g., weeks or months or years). Implantable medical devices can include devices configured for bioabsorbtion within a body during a prolonged period of time.

As used herein, “endolumenal” or “translumenal” refer to a device adapted for placement within a body vessel by procedures wherein the prosthesis is advanced within and through the lumen of a body vessel from a remote location to a target site within the body vessel. In vascular procedures, a medical device can typically be introduced “endovascularly” using a catheter over a guidewire under fluoroscopic guidance. The catheters and guidewires may be introduced through conventional access sites to the vascular system, such as through the femoral artery, or brachial and subclavian arteries, for access to the coronary arteries.

As used herein, the term “body vessel” means any body passage lumen that conducts fluid, including but not limited to blood vessels, esophageal, intestinal, billiary, urethral and ureteral passages.

The term “bioabsorbable” is used herein to refer to materials selected to dissipate upon implantation within a body, independent of which mechanisms by which dissipation can occur, such as dissolution, degradation, absorption and excretion. The terms “bioabsorbable,” “bioresorbable,” or “biodegradable” are used synonymously herein, unless otherwise specified, to refer to the ability of the material or its degradation products to be removed by biological events, such as by fluid transport away from the site of implantation or by cellular activity (e.g., phagocytosis). Only the term “bioabsorbable” will be used in the following description to encompass absorbable, absorbable, bioabsorbable, and biodegradable, without implying the exclusion of the other classes of materials.

As used herein, recitation of a “non-bioabsorbable” material refers to a material, such as a polymer or copolymer, which remains in the body without substantial bioabsorption.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The term “mixture” refers to a combination of two or more substances in which each substance retains its own chemical identity and properties.

The terms “frame” and “support frame” are used interchangeably herein to refer to a structure that can be implanted, or adapted for implantation, within the lumen of a body vessel. In one embodiment, the frame may function as a stent.

As used herein, a “stent” is any structure that is used to hold tissue in place within a body, including an interior portion of a blood vessel, lymph vessel, ureter, bile duct or portion of the alimentary canal. A stent may be useful for opening up blood vessels, such as for example, an artery, vein or capillary thereby improving blood flow; keeping an artery, vein or capillary open; sealing any tears or openings in an artery, vein or capillary; preventing an artery, vein or capillary wall from collapsing or closing off again; or preventing small pieces of plaque from breaking off. In one embodiment, the stent is a stent graft.

A “stent graft,” as used herein, refers to a support frame attached to a graft material. A stent graft can be any stent that is covered with a synthetic or natural (i.e., biologically-derived) material to form a graft prosthesis. The term also encompasses grafted stents, wherein the stent is covered in its entirety with a natural or synthetic graft material (e.g., Vanguard-graft stent, Palmaz-Impragraft stent or Corvita stent). In one embodiment, the stent graft is a prosthetic.

The term “graft material” as used herein refers to a flexible material that can be attached to a support frame, for example to form a stent graft. A graft material can have any suitable shape, but preferably forms a tubular prosthetic vessel. A graft material can be formed from any suitable material, including the biologically derived or synthetic materials described herein.

The term “elastin-stabilizing compound” refers to a compound or a plurality of compounds, chemical compositions, polymers, polypeptides, polynucleotides, etc. that is capable of interacting with elastin, and in so doing rendering elastin at least partially resistant to physical and biological degradation. Elastin-stabilizing compounds include phenolic compounds.

The term “phenolic compounds” refers to a compound or a plurality of compounds, chemical compositions, polymers, polypeptides, polynucleotides, etc. that include at least one phenolic group. One example of a phenolic compound is a phenolic tannin. Phenolic tannins are described in more detail below.

The term “pharmaceutically acceptable carrier” or “carrier” includes any material which, when combined with phenolic tannin, allows the tannin compound to retain biological activity, such as the ability to bind and stabilize elastin in the host connective tissue, and is non-reactive with the subject's immune system. Examples include, but are not limited to, any of the standard pharmaceutical carriers such as a phosphate buffered saline solution, water, emulsions such as oil/water emulsions, various polymer carrier materials, and various types of wetting agents. Compositions comprising such carriers are formulated by well known conventional methods (see, for example, Remington's Pharmaceutical Sciences, Chapter 43, 14th Ed., Mack Publishing Co., Easton, Pa.).

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response, and is distinguished from a mild, transient inflammation which typically accompanies surgery or implantation of foreign objects into a living organism.

The term “coating,” as used herein and unless otherwise indicated, refers generally to material attached to a medical device. A coating can include material covering any portion of a medical device, and can be configured as one or more coating layers. A coating can have a substantially constant or a varied thickness and composition. Coatings can be adhered to any portion of a medical device surface, including the lumenal surface, the ablumenal surface, or any portions or combinations thereof.

As used herein, the phrase “controlled release” refers to the release of a therapeutic compound at a predetermined rate. A controlled release may be characterized by a drug elution profile, which shows the measured rate that the material is removed from a material-coated device in a given solvent environment as a function of time. A controlled release does not preclude an initial burst release associated with the deployment of the medical device, because in some embodiments of the invention an initial burst, followed by a more gradual subsequent release, may be desirable. The release may be a gradient release in which the concentration of the therapeutic compound released varies over time or a steady state release in which the therapeutic compound is released in equal amounts over a certain period of time (with or without an initial burst release).

When coated, the coating may be present on any portion of a surface of the device. In one embodiment, the surface is the inner surface. In another embodiment, the surface is the outer surface. In one embodiment, the layer covers at least about 10% of the surface. In another embodiment, the layer covers at least about 20% of the surface. In another embodiment, the layer covers at least about 30% of the surface. In another embodiment, the layer covers at least about 40% of the surface. In another embodiment, the layer covers at least about 50% of the surface. In another embodiment, the layer covers at least about 60% of the surface. In another embodiment, the layer covers at least about 70% of the surface. In another embodiment, the layer covers at least about 80% of the surface. In another embodiment, the layer covers at least about 90% of the surface. In another embodiment, the layer covers about 100% of the surface.

As used herein, the term “preventing” includes inhibiting an aortic aneurysm and/or aortic dissection, in particular, abdominal aortic aneurysm and abdominal aortic dissection.

As used herein, the term “treating” includes eradicating an aortic aneurysm and/or aortic dissection, in particular, abdominal aortic aneurysm and abdominal aortic dissection. In one embodiment, “treating” refers to minimizing the spread or minimizing the worsening of a aortic aneurysm and/or aortic dissection, in particular, an abdominal aortic aneurysm and abdominal aortic dissection.

The term “therapeutically effective amount” refers to a dose of a compound, chemical compositions, polymers, polypeptides, polynucleotides, etc. that effectively prevents degradation of components of connective tissue, such as elastin, but does not cause undesirable or intolerable side effects.

As used herein, the term “patient” means an animal (e.g., cow, horse, sheep, pig, chicken, turkey, quail, cat, dog, mouse, rat, rabbit or guinea pig), preferably a mammal such as a non-primate or a primate (e.g., monkey or human), most preferably a human.

Elastin-Stabilizing Compounds

The present invention provides local delivery of one or more elastin-stabilizing compounds proximate to a site of treatment within a body vessel by a medical device. In one embodiment, the elastin-stabilizing compound may be a phenolic compound. In another embodiment, the elastin-stabilizing compound may be a phenolic tannin compound. One or more phenolic tannin compounds may be provided for release from the medical device. The phenolic tannin compound(s) may, for example, be included as part of at least a portion of the base material of the medical device itself; be contained within a reservoir, a well or a groove; be within a carrier material deposited on at least a portion of the medical device, or as a separate layer deposited on at least a portion of the medical device (the layer may optionally be over coated with another layer) or on at least a portion of the medical device that has been coated with a primer layer for increased adhesion; or within the hollow walls of the device; or any combination of these. The phenolic tannin compound may also be included in a separate carrier layer (or a multi-layered structure) that may be placed between elements of the medical device. For example, the separate layer may be placed between a stent and a graft material. In certain embodiments, the release of the phenolic tannin compound from the medical device depends, in part, upon the composition and configuration of the carrier material and/or the coating layer(s).

Phenolic Compounds

Either a single phenolic compound or a combination of phenolic compounds may be used in the present invention. Consequently, as used herein and in the appended claims, and as noted above, the term “phenolic compound” refers to either a single compound or a combination of compounds that include at least one phenolic group.

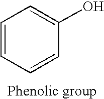

When more than one phenolic group is present, these compounds are called polyphenols. Polyphenols comprise one category of plant secondary metabolites.

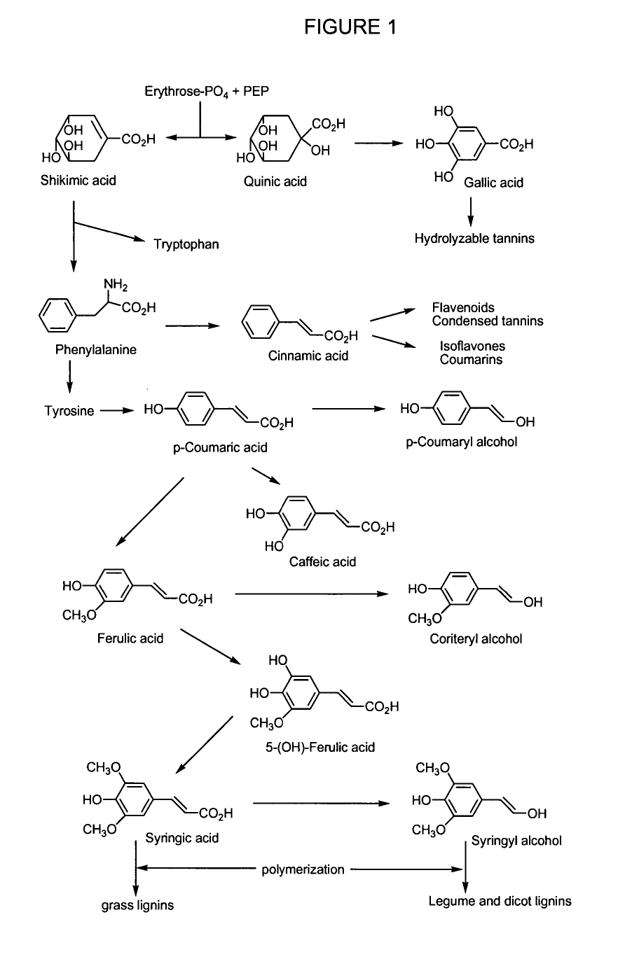

All phenolic compounds may be formed either directly or indirectly via the Phenylpropanoic or Shikimic acid pathway, shown schematically in FIG. 1. Exemplary phenolic compounds formed from this pathway include: aromatic amino-acids, isoflavones, lignins, coumarins and tannins. Phenolic tannins are preferred compounds for use in this invention.

Phenolic Tannins

The term “phenolic tannins” refers to a broad group of plant (poly)phenolic compounds with multiple structure units with free phenolic groups. These compounds may have a surprising ability of stabilizing components of the connective tissue, such as elastin and collagen. Phenolic tannins may be stabilizing elastin by the process of cross-linking. “Cross-linking” refers generally to the process of forming bonds, e.g., covalent bonds or hydrogen bonds, between free, active moieties on or within tissue or between a cross-linking agent or other compound which reacts with a reactive moiety of the tissue. Specifically, phenolic tannins may form multiple hydrogen bonds with proteins, such as elastin and collagen, which are particularly rich in proline residues. The process of cross-linking stabilizes these proteins so as be less antigenic and less susceptible to physical and biological (e.g., enzymatic) degradation. Phenolic tannin compounds may have this surprising ability and are preferred compounds for use in this invention. Other compounds capable of binding to or cross-linking elastin, and in so doing rendering elastin at least partially resistant to degradation, are also contemplated for use in accordance with this invention.

Without being limited to a particular theory of biological function, recent studies have suggested that phenolic tannin compounds may bind elastin in a time-dependent fashion, increasing the resistance of the elastin to enzymatic degradation. For example, Isenburg et al. have reported treatment of lyophilized aortic elastin strips in buffered solutions of 0.3%-0.8% tannic acid reduced the susceptibility of the elastin towards elastase by about 50%, and elastin stabilization in ascending porcine aorta at tannic concentrations of about 0.3% (“Elastin stabilization in cardiovascular implants: improved resistance to enzymatic degradation by treatment with Tannic acid”, Isenburg, J. C. et al., Biomaterials, 25, 2004 3293-3302; “Tannic acid treatment enhances biostability and reduces calcification of Glutaraldehyde fixed aortic wall”, Isenburg, J. C. et al., Biomaterials 26, 2005, 1237-1245). Other reports indicated that treatment of human peripheral blood mononucleocytes exposed to gallotannin beta-D-pentagalloylglucose (beta-PGG) (a central component of tannic acid) decreases tumor necrosis factor-alpha (TNF-alpha) output from human peripheral blood mononucleocytes exposed to lipopolysaccharide (LPS) by as much as 90% (vs. control) at approximately 5 uM concentration. “In vitro and in vivo inhibition of LPS-stimulated tumor necrosis factor-alpha secretion by the gallotannin beta-D-pentagalloylglucose” Bioorg Med Chem Lett. 2001; 11(14):1813-5, Feldman K S, Sahasrabudhe K, Lawlor M D, Wilson S L, Lang C H, Scheuchenzuber W J. A qualitatively similar but less pronounced effect (approximately 50% decrease) was observed in the serum of rats dosed with both LPS and beta-PGG. “In vitro and in vivo inhibition of LPS-stimulated tumor necrosis factor-alpha secretion by the gallotannin beta-D-pentagalloylglucose” Bioorg Med Chem Lett. 2001; 11(14):1813-5, Feldman K S, Sahasrabudhe K, Lawlor M D, Wilson S L, Lang C H, Scheuchenzuber W J.

Phenolic tannins have been previously suggested as fixatives for cardiac prostheses such as heart valves, other replacement heart components, and cardiac vascular grafts. Use of phenolic tannins as fixatives was described in U.S. Publication No. 2004/0153145 A1, published on Aug. 5, 2004, which is incorporated by reference herein in its entirety.

Phenolic tannins may be classified according to some of their chemical characteristics and include hydrolyzable tannins and condensed tannins. Typically, a hydrolyzable tannin has at least 3-OH groups of the glucose moiety esterified to exhibit a sufficiently strong binding capacity.

All the tannins described below may be used in accordance with this invention. Other suitable phenolic tannins, derivatives and mixtures thereof, will be known to those of ordinary skill in the art and are also included.

1. Hydrolyzable Tannins

Hydrolyzable tannins are polyphenols with a polyol (poly alcohol, many —OH groups) as a central core. Usually this polyol molecule is D-glucose. In hydrolyzable tannins the —OH groups may be esterified (partial or totally) with phenolic groups. Each hydrolyzable tannin molecule may thus be composed of a core of β-D-Glucose and 6 to 14 galloyl groups. A structure of one isomer of tannic acid is shown in Table A below.

TABLE A

| Com- | | |
|---|---|---|
| pound | Name | Structure |
| 1 | Tannic acid | 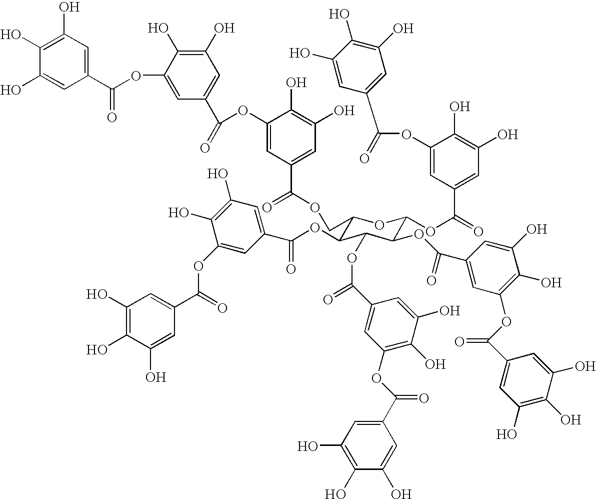 |

Hydrolyzable phenolic tannins may have molecular weight ranging from about 500 to>about 200,000 weight average and are soluble in water, with exception of some high molecular weight structures. As mentioned above, all phenolic tannins have the ability to bind proteins and form insoluble or soluble tannin-protein complexes.

Early work on hydrolyzable tannins included Haslam's significant elucidations of the structures of the simple gallotannins (Haslam, E. Plant polyphenols. Vegetable tannins revisited, ed.; Cambridge University Press: Cambridge, U. K., 1989). More recently, Okuda et al. (Okuda, T. et al. Hydrolyzable tannins and related polyphenols. Progress in the Chemistry of Organic Natural Products 1995, 66:1-117) characterized and classified complex hydrolyzable tannins.

Hydrolyzable phenolic tannin compounds according to the present invention include tannic acid and other tannin compounds, such as gallotannins, ellagitannins, taragallotannins, caffetannins, as well as derivatives and mixtures thereof.

A. Gallotannins

Gallotannins are the simplest hydrolyzable tannins, which are simple polygalloyl esters of glucose. The prototypical gallotannin is pentagalloyl glucose (β-1,2,3,4,6-Pentagalloyl-O-D-Glucopyranose shown in Table B below). Pentagalloyl glucose, or PGG, has five identical ester linkages with phenolic groups, such as gallic acid, that involve aliphatic hydroxyl groups of the core sugar. PGG is one, especially preferred tannin for use in this invention.

TABLE B

| Com- | | |
|---|---|---|
| pound | Name | Structure |
| 2 | β-1,2,3,4,6- Pentagalloyl-O- D- Glucopyranose | 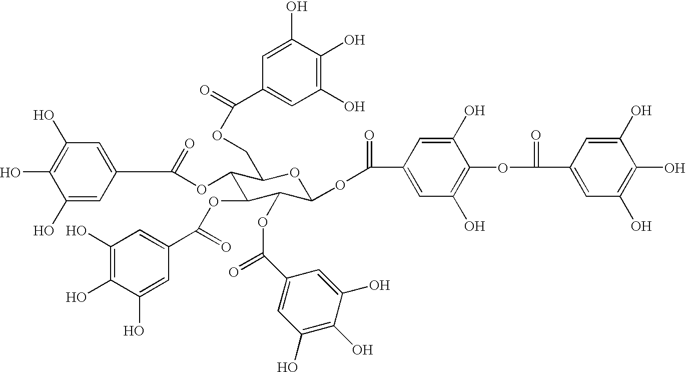 |

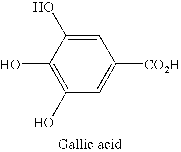

Like all of the gallotannins, PGG has many isomers. The molecular weights of all the isomers are the same (940 g/mol), but chemical properties, such as susceptibility to hydrolysis and chromatographic behavior, and biochemical properties, such as ability to precipitate proteins, are structure dependent. A structure of an exemplary isomer of PGG is shown in Table C.

TABLE C

| Com- | | |
|---|---|---|
| pound | Name | Structure |
| 3 | β-1,2,2,3,6-Pentagalloyl- O-D-glucose | 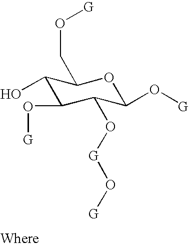 |
| | | 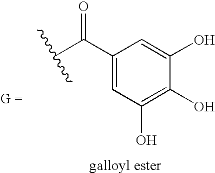 |
| | | 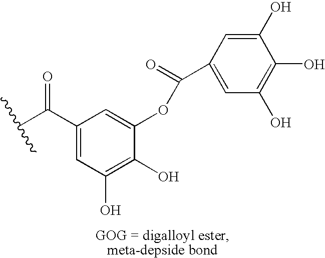 |
| | | 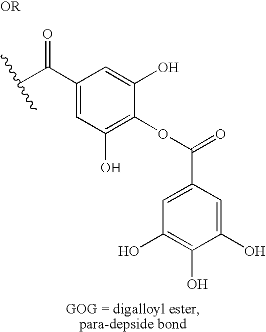 |

The polygalloyl ester chains found in gallotannins may be formed by either meta- or para-depside bonds, involving a phenolic hydroxyl rather than an aliphatic hydroxyl group.

Simple gallotannins, up to 12 esterified galloyl groups and a core glucose, may be routinely found in tannins from sumac or oak galls. Gallotannins may also be obtained from tannic acid obtained from the twig galls of sumac, Rhus semialataor or oak galls.

Tannic acid may be naturally derived polyphenol that can cross-link proteins by the formation of multiple hydrogen bonds. It has a penta galloyl-D-glucose core and five more units of galloyl linked to one of the galloyl of the core. Additional properties of tannic acid may be found in reference to the publication by Haslam, E. Plant Polyphenols, Cambridge University Press, Cambridge U.K., 1989, pp. 123-195, which is incorporated herein by reference.

Commercial tannic acid is composed of mixtures of gallotannins from sumac galls (Chinese gallotannin); Aleppo oak (Quercus infectoria) galls (Turkish gallotannin); or sumac (R. coriaria, R. typhina) leaves (sumac gallotannin). Although commercial sources provide a molecular weight for tannic acid (1294 g/mol), the preparations are heterogenous and contain variable mixtures of galloyl esters.

Tannic acid is known to cross-link with collagen. In addition, tannic acid has been used as an elastin stain for electron microscopy, and has been used as a contrast-increasing agent for collagen staining. Additionally, tannic acid is known to have antibacterial properties, can inhibit enzymes, and can reduce protein antigenicity.

Most importantly, tannic acid can interact with elastin as well as other connective tissue components. For instance, tannic acid may be capable of cross-linking glycosaminoglycan polysaccharides and other connective tissue components. Specifically, tannic acid is believed able to interact with elastin through proline-rich areas within the elastin matrix molecules.

PGG can be prepared from some commercial tannic acids by methanolysis in acetate buffer. Some commercial tannic acid preparations include Chinese or sumac gallotannin. Turkish gallotannin is composed of esters of 1,2,3,6-tetragalloyl glucose; or 1,3,4,6-tetragalloyl glucose, which are shown in Table D.

TABLE D

| Com- | | |
|---|---|---|
| pound | Name | Structure |
| 4 | 1,2,3,6-tetragalloyl glucose | 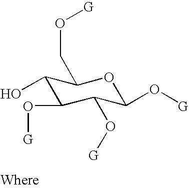 |
| | | 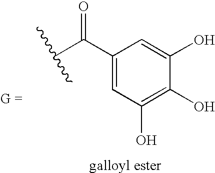 |
| 5 | 1,3,4,6-tetragalloyl glucose | 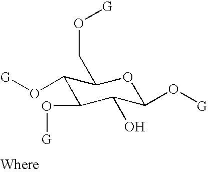 |
| | | 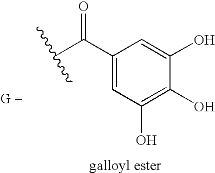 |

Although for many gallotannins glucose is the alcohol, other polyols including glucitol, hammamelose, shikimic acid, quinic acid, and quercitol, have been reported as constituents of gallotannins from a few species. The structures of these polyols are shown below.

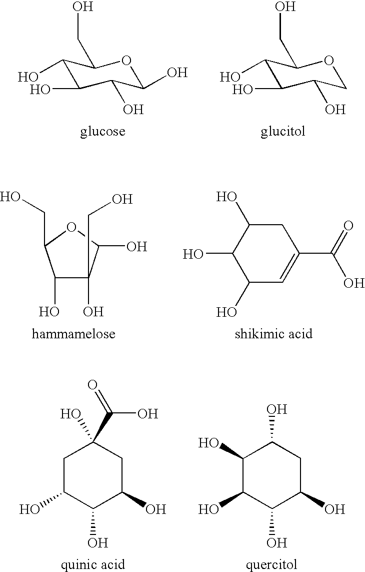

For example, aceritannin that includes glucitol as the polyol is found in leaves of several species of maple (Acer), and hamamelitannin, which includes hammamelose as the polyol is found in bark of witch hazel (Hamamelis virginiana), oak (Quercus rubra), and several chestnut species (Castanea sp.). Structures of these exemplary gallotannins are shown in Table E.

TABLE E

| Com- | | |
|---|---|---|
| pound | Name | Structure |
| 6 | Aceritannin = 2,6-di-O- galloyl-1,5-anhydro-D- glucitol | 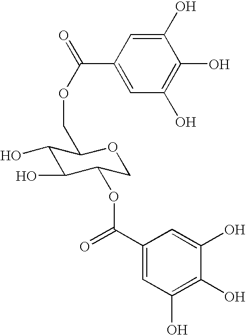 |
| 7 | Hamamelitannin | 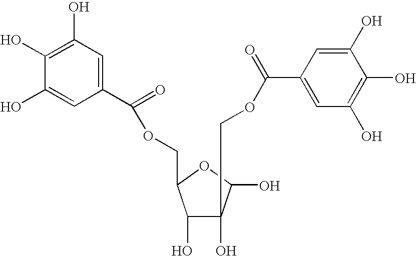 |

B. Ellagitannins

Ellagitannins are phenolic tannins that include phenolic groups that consist of hexahydroxydiphenic acid (HHDP), which may spontaneously dehydrate to the lactone form, ellagic acid.

Ellagitannins have molecular weight ranging from about 2000 to about 5000 weight average and may be hydrolyzed by mild acids or mild bases to yield carbohydrate and phenolic acids. A structure of an exemplary ellagitannin is shown in Table F below.

TABLE F

| Com- | | |
|---|---|---|
| pound | Name | Structure |
| 8 | Ellagitannin | 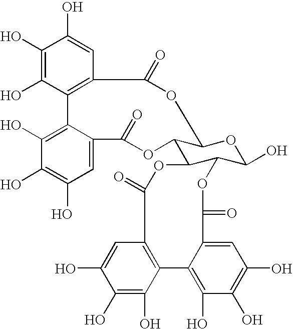 |

Ellagitannins may be formed by oxidative coupling of galloyl groups, as illustrated below.

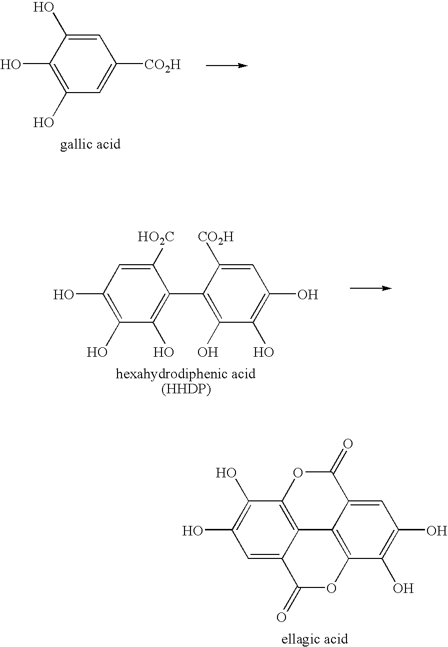

Intramolecular carbon-carbon coupling to form HHDP is most common between C-4/C-6 (e.g., eugeniin); and C-2/C-3 (e.g., casuarictin, also has C-4/C-6), as would be expected for polygalloyl glucoses in the more stable ⁴C₁ conformation. However, in a few plants intramolecular coupling occurs at C-3/C-6 (e.g., corilagin), C-2/C-4 (e.g., geraniin, also has C-3/C-6), or C-1/C-6 (e.g., davidiin), suggesting the polygalloyl glucose starting material was in the less stable ⁴C₁ conformation. Geraniin is further characterized by partial oxidation of the C-2/C-4 HHDP to dehydro-HHDP, and in aqueous solution several forms of dehydro-HHDP can be detected in geraniin by NMR. Structures of these tannins are shown in TABLE G.

TABLE G

| Com- | | |
|---|---|---|
| pound | Name | Structure |
| 9 | Eugeniin | 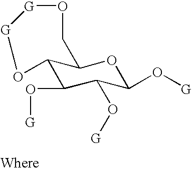 |
| | | 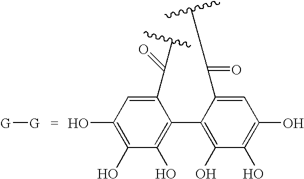 |
| 10 | Casuarictin | 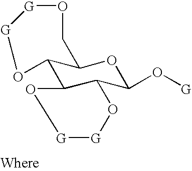 |
| | | 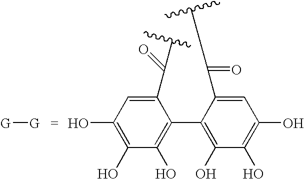 |
| 11 | Corilagin | 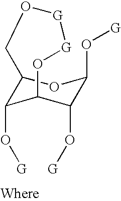 |
| | | 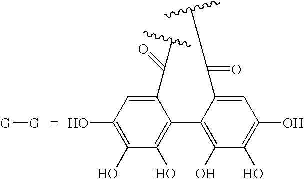 |
| 12 | Geraniin | 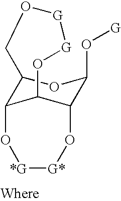 |
| | | 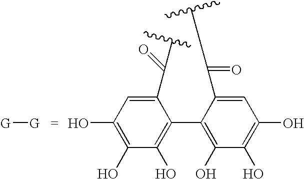 |
| | | 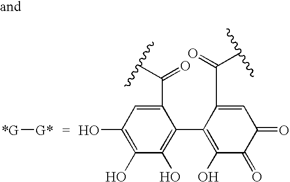 |
| 13 | Davidiin | 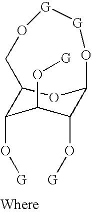 |
| | | 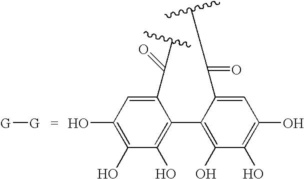 |

In some plants, including oak and chestnut, the ellagitannins are further elaborated via ring opening. Thus, after conversion of casuariin, casuarinin, castalagin, and castling; stachyurin, vescalagin, and vescalin forms. Structures of castalagin and vescalagin are shown in Table H.

TABLE H

| Com- | | |
|---|---|---|
| pound | Name | Structure |
| 14 | Castalagin | 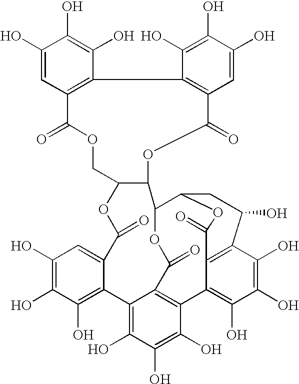 |
| 15 | Vescalagin | 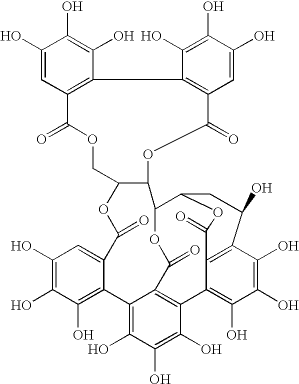 |

The ellagitannins can undergo intermolecular oxidative coupling with other hydrolyzable tannins to yield dimers. For example, in several euforbs (e.g., Euphorbia watanabei) geraniin oxidatively condenses PGG to yield various euphrobins, characterized by valoneoyl group. An exemplary structure of and euphorbin is shown in Table I.

TABLE I

| Com- | | |
|---|---|---|
| pound | Name | Structure |
| 16 | Euphorbin | 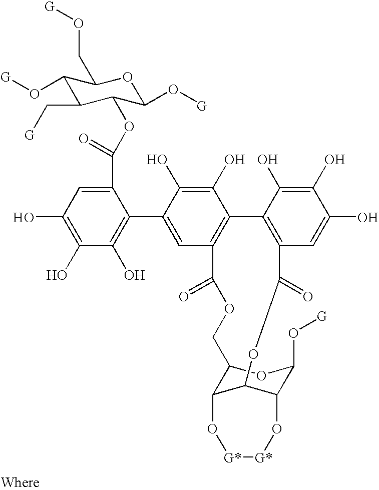 |
| | | 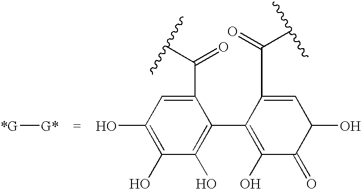 |

Additional examples of tannins include Oenothein B, Woodfordin C, Cuphiin D₁, and Eugeniflorin D₁, which are macrocyclic dimers linked by two valoneoyl groups, and the nobotanins are macrocyclic trimers. Structure of Oenothein B is shown in Table J below.

TABLE J

| Com- | | |
|---|---|---|
| pound | Name | Structure |
| 17 | Oenethein B | 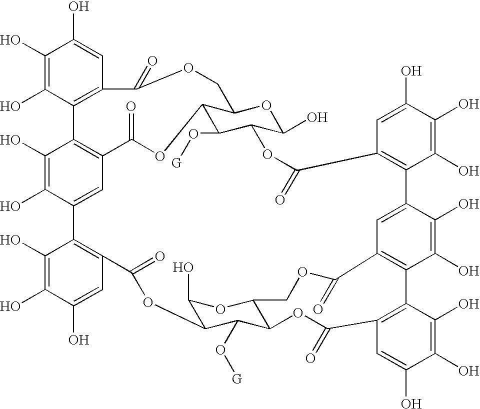 |

2. Condensed Tannins

Condensed tannins or Proanthocyanidins are defined as oligo or polymers of flavonoid (flavan-3-ol) linked by carbon-carbon bonds not susceptible to hydrolysis. The flavonoids are a diverse group of metabolites based on a heterocyclic ring system derived from phenylalanine (B) and polyketide biosynthesis (A). The flavonoid skeleton, the standard letters to identify the rings, and the numbering system are shown in the diagram below.

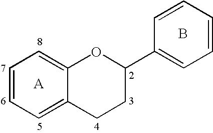

Generally, an example of a condensed tannin is shown below.

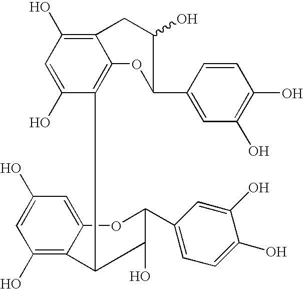

Condensed tannins may be based on the flavan-3-ols (−)-epicatechin and (+)-catechin. Addition of third phenolic group on the B ring yields epigallocatechin and gallocatechin. Much less common are flavan-3-ols with only a single phenolic group on the B ring, para to C-2 (epiafzelechin, afzelechin with stereochemistry corresponding to epicatechin, catechin respectively).

Exemplary Flavan-3-ols are shown in Table K below.

TABLE K

| Com- | | |
|---|---|---|
| pound | Name | Structure |
| 18 | Epicatechenin | 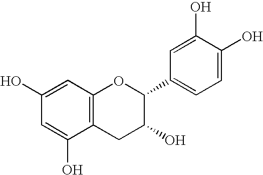 |
| 19 | Catechin | 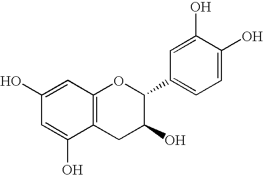 |
| 20 | Epigallocatechin | 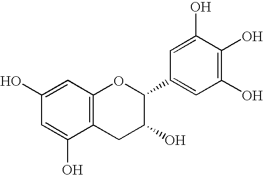 |
| 21 | Gallocatechin | 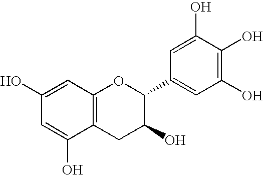 |
| 22 | Epiafzelechin | 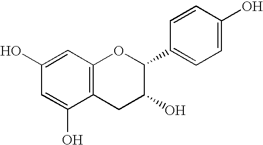 |
| 23 | Afzelechin | 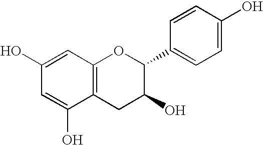 |

Some condensed tannins may be linked via a carbon-carbon bond between C8 of the terminal unit and C4 of the extender. The four common modes of coupling are illustrated by dimers shown in Table L. In addition to these dimers, related dimers linked by C6 of the terminal unit and C4 of the extender have been isolated and may be used in this invention.

TABLE L

| Com- | | |
|---|---|---|
| pound | Name | Structure |
| 24 | Epicatechin (4β->8)- catechin | 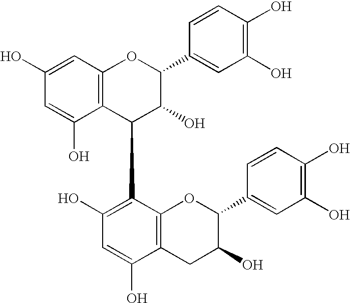 |
| 25 | Epicatechin (4β->8)- epicatechin | 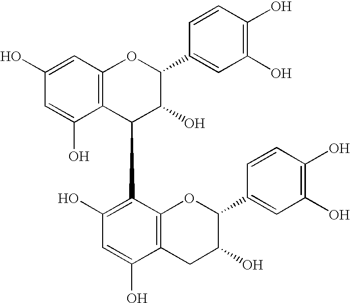 |
| 26 | Catechin (4α->8)- catechin | 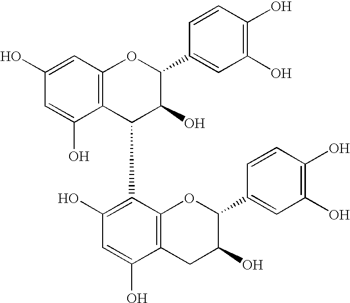 |
| 27 | Catechin (4α->8)- epicatechin | 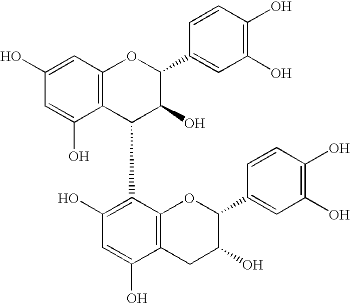 |

Further polymerization can yield the linear 4,8 polymers such as the Sorghum procyanidin, structure of which is shown in Table M. Linear polymers based on 4,6, dimers; and branched dimers containing both 4,6 and 4,8 linkages are also contemplated for use in this invention.

TABLE M

| Com- | | |
|---|---|---|
| pound | Name | Structure |
| 28 | Sorghum procyanidin; i.e., epicatechin-[(4b->8)- epicatechin]15-(4b->8)- catechin | 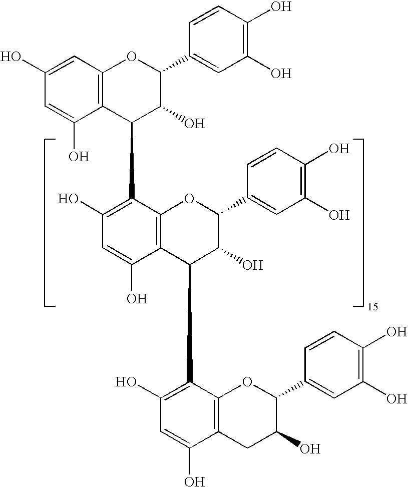 |

Although the term condensed tannins is still widely used to describe the flavonoid-based polyphenolics, as mentioned above, the chemically more descriptive term “proanthocyanidins” may also be used to refer to condensed tannins. The term proanthocyanidins is related to the oxidation reaction of acidic alcohol solutions (oxidative cleavage and NOT hydrolysis) under heating that produce red anthocyanidins. Anthocyanidins give red-blue colors to leaves, flowers and fruits as well as the sensorial astringency.

The products of the acid butanol reaction are an unmodified terminal unit, and the colored anthocyanidins produced by the extender units. The general formula of anthrocyanidin is shown below.

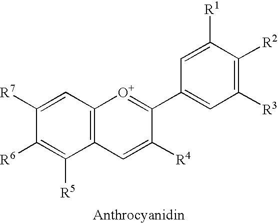

Catechin- and epicatechin-based polymers produce cyanidin, and thus are known as procyanidins. Gallocatechin and epigallocatechin-based polymers yield delphinidin, and the rare mono-substituted flavan-3-ol based polymers yield pelargonidin. The structures of these anthocyanidins tannins and others are shown in Table N below.

TABLE N

| Com- | | |
|---|---|---|
| pound | Name | Structure |
| 29 | Aurantinidin | 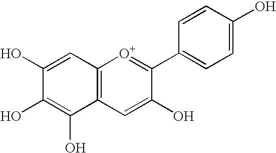 |
| 30 | Cyanidin | 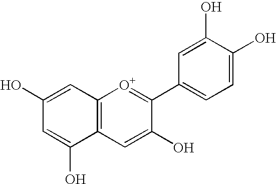 |
| 31 | Delphinidin | 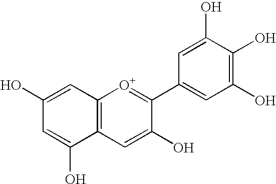 |
| 32 | Europinidin | 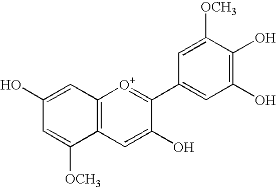 |
| 33 | Luteolinidin | 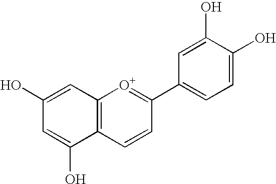 |
| 34 | Pelargonidin | 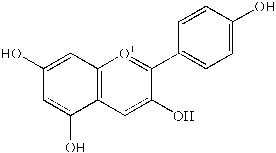 |
| 35 | Malvidin | 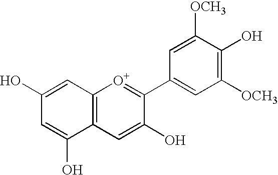 |
| 36 | Peonidin | 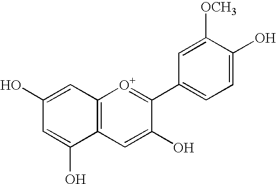 |
| 37 | Petunidin | 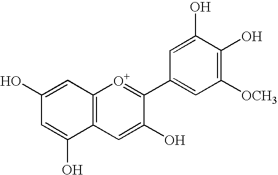 |
| 38 | Apigeninidin | 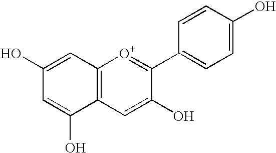 |
| 39 | Robinetinidin | 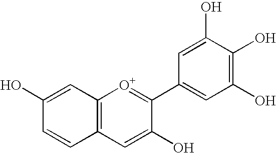 |
| 40 | Fisetinidin | 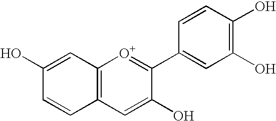 |
| 41 | Guibourtinidin | 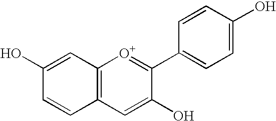 |

Another important group of condensed tannins are 5-deoxy-flavan-3-ols polymers. Branching is common in these tannins because of the reactivity of the 5-deoxy A ring. Profisetinidins and prorobinetinidins comprise the major tannins found in quebracho and acacia tannin preparations. Acid butanol reaction yields the 5-deoxy anthcyanidins fisetinidin and robinetidin, structures of which are shown in Table O below.

TABLE O

| Com- | | |
|---|---|---|
| pound | Name | Structure |
| 42 | Robinetinidol-(4α->8)- catechin-(6α->4a)- robinetinidol | 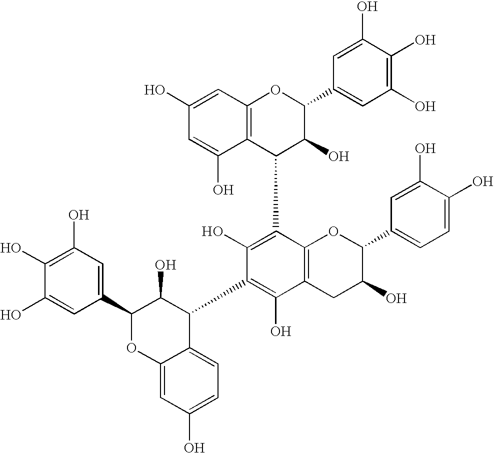 |
| 43 | Profisetinidin | 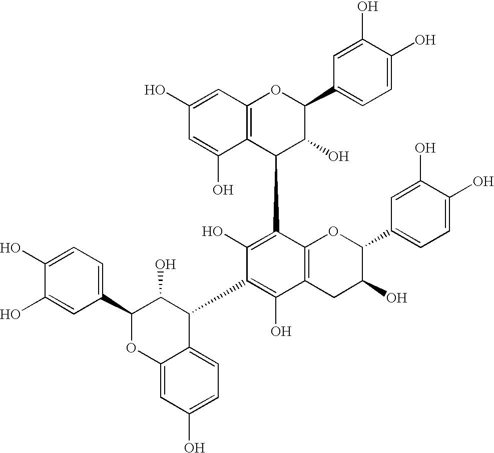 |

Another type of linkage that has been described involves oxidative C—O coupling between the flavonoid rings to yield epicatechin (2β-->7,4β-->8)-epicatechin, structure of which is shown in Table P, and related proanthocyanidins.

TABLE P

| Com- | | |
|---|---|---|
| pound | Name | Structure |
| 44 | Epicatechin (2β-->7,4β-- >8)-epicatechin | 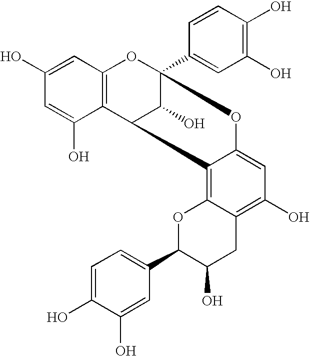 |

The flavan-3,4-diols, or luecoanthocyanidins, which are monomeric flavonoids that have reactive chemistry similar to that of condensed tannins described below are also contemplated for use in this invention. Structures of some exemplary flavan-3,4-diols are shown in Table R below.

TABLE R

| Com- | | |
|---|---|---|
| pound | Name | Structure |
| 45 | Leucofisetinidin | 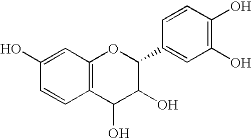 |
| 46 | Leucopelargonidin | 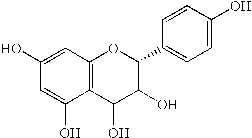 |
| 47 | Leucocyanidin | 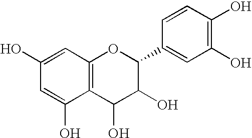 |
| 48 | Leucodelphinidin | 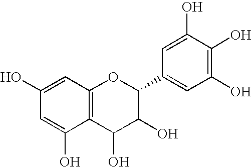 |

Condensed tannins also include flavan-4-ols, which are also luecoanthocyanidins. Structures of some exemplary flavan-4-ols are shown in Table S below.

TABLE S

| Com- | | |
|---|---|---|
| pound | Name | Structure |
| 49 | Leucoapigeninidin | 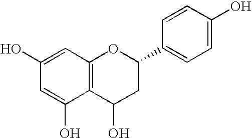 |
| 50 | Leucoluteolinidin | 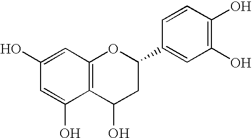 |

Condensed tannins are widely distributed among many different plant families of economic importance, such as Leguminoseae (soybeans, beans, many tropical forages, etc.), Myrtaceae (Eucalyptus sp. Mirtus sp.), Anacardiaceae (Quebracho, Scinopsis balansae). Oaks, Maples, Pines, and Sorghums are some known species rich in condensed tannins.

All the tannins described above may be used in accordance with this invention. Other suitable phenolic tannins, derivatives, or mixtures thereof, will be known to those of ordinary skill in the art and are also included.

In one example, phenolic tannins may be, for example, admixed with excipients or carriers suitable for either enteral or parenteral application. In one embodiment, phenolic tannins may be admixed with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; and/or if desired c) disintegrants, e.g., starches, agar, alginic acid or its sodium salt, or effervescent mixtures. These compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The phenolic tannin compositions may be prepared according to conventional mixing, granulating or coating methods, respectively. The phenolic tannin compositions may contain about 0.1 to 95% of the phenolic tannin. In another embodiment, the phenolic tannin compositions may contain about 0.1 to 75% of the phenolic tannin. In yet another embodiment, the phenolic tannin compositions may contain about 1 to 50% of the phenolic tannin.

Methods of Treatment

In one embodiment, the present invention provides a method for treating a patient having an aneurysm, and especially abdominal aortic aneurysm, the method comprising a step of delivering a medical device of this invention and as described below to a point of treatment within the patient having the aneurysm. The medical device is adapted to release the elastin-stabilizing compound at the point of treatment within the body lumen of the patient.

In another aspect, the present invention provides a method for treating a patient having an aortic dissection, the method comprising the step of delivering a medical device and elastin-stabilizing compound of this invention and as described below to the patient at a point of treatment within the patient having the aortic dissection. The medical device is adapted to release the elastin-stabilizing compound at or near the point of treatment within the body lumen of the patient.

For example, the medical device can release or retain an elastin-stabilizing compound at a desired rate within a blood vessel upon placement proximate to an aneurysm or aortic dissection. By providing elastin-stabilizing compound with the device, the progression of local endovascular disease or aortic dissection may be mitigated, stopped and/or reversed, preventing further weakening and dilation of the vessel wall or splitting of the layers of aorta.

In another aspect, the present invention provides a method of treating an aneurysm or an aortic dissection comprising radially expanding a medical device and elastin-stabilizing compound of this invention and as described below, in a lumen with a balloon catheter, wherein the balloon catheter releases an elastin-stabilizing compound.

In yet another aspect, the present invention is a method of treating an aneurysm or an aortic dissection comprising radially expanding a balloon catheter comprising an elastin-stabilizing compound in a lumen, wherein the balloon catheter releases the elastin-stabilizing compound within the lumen.

Medical Devices Comprising Elastin-Stabilizing Compounds

In another embodiment, the invention provides a medical device and one or more elastin-stabilizing compounds. In one embodiment, the medical device may be an implantable device. As mentioned above, the elastin-stabilizing compounds may be phenolic compounds, such as phenolic tannins.

In one embodiment, a therapeutically effective concentration of elastin-stabilizing compound can be incorporated in the medical device. The concentration of the elastin stabilizing compound per unit of ablumenal surface area of the medical device can be selected to achieve a desired tissue concentration upon implantation of the medical device. A therapeutically effective amount of elastin stabilizing compound can be selected based on considerations such as the material of the medical device surface, the design of the medical device, the coating configuration and the molecular structure of the elastin stabilizing compound, all of which can determine the rate of elution of the elastin-stabilizing compound within a particular body vessel. In one instance, the elastin-stabilizing compound is incorporated in the medical device so that from about 0.05 mg/g to about 50 mg/g of the elastin-stabilizing compound is delivered to the affected tissue lining the wall of a body vessel proximate to the medical device. In another instance, the elastin-stabilizing compound is incorporated in the medical device so that from about 0.1 mg/g to about 10 mg/g of the elastin-stabilizing compound is delivered to the affected tissue lining the wall of a body vessel proximate to the medical device. In yet another instance, the elastin-stabilizing compound is incorporated in the medical device so that from about 0.5 mg/g to about 5 mg/g of elastin-stabilizing compound is delivered to the tissue.

In one embodiment, the elastin-stabilizing compound may be coated on the ablumenal surface of the medical device in an amount effective to increase resistance of elastin in tissue proximate to the ablumenal surface to enzymatic degradation, for example by elastase. In another embodiment, phenolic tannin compounds may be coated on the ablumenal surface at concentrations sufficient to deliver a desired amount of the phenolic tannin compound for increasing resistance of elastin in an adjacent body vessel site to degradation. For instance, a phenolic tannin compound may be included on the ablumenal surface of a medical device at a concentration effective to deliver from about 0.05 mg/g to about 50 mg/g of the phenolic tannin compound to adjoining body vessel wall tissue upon placement of the medical device within the body vessel lumen. Alternatively, a phenolic tannin compound may be included on the ablumenal surface of a medical device at a concentration effective to deliver from about 0.1 mg/g to about 10 mg/g of the phenolic tannin compound to adjoining body vessel wall tissue upon placement of the medical device within the body vessel lumen. In another instance, a phenolic tannin compound may be included on the ablumenal surface of a medical device at a concentration effective to deliver from about 0.5 mg/g to about 5 mg/g of the phenolic tannin compound to adjoining body vessel wall tissue upon placement of the medical device within the body vessel lumen.

The medical device can be adapted to release a phenolic tannin compound at a predetermined location within body of a patient. The medical device can have any suitable configuration. In one embodiment, the medical device may be an implantable medical device, such as graft, stent, and stent graft. The implantable medical device may be an endolumenal medical device, which may be placed inside a lumen of a patient. For example, a stent graft may be placed inside a body vessel. Alternatively, an implantable device may be a medical device, which may be placed on the outside of a body lumen during an open surgery. For example, a vascular wrap comprising an elastin-stabilizing compound may be placed on the outside of the vessel. In yet another embodiment, the medical device may be a delivery device, such as a balloon catheter. Exemplary medical devices are described below. Other configurations are also contemplated.

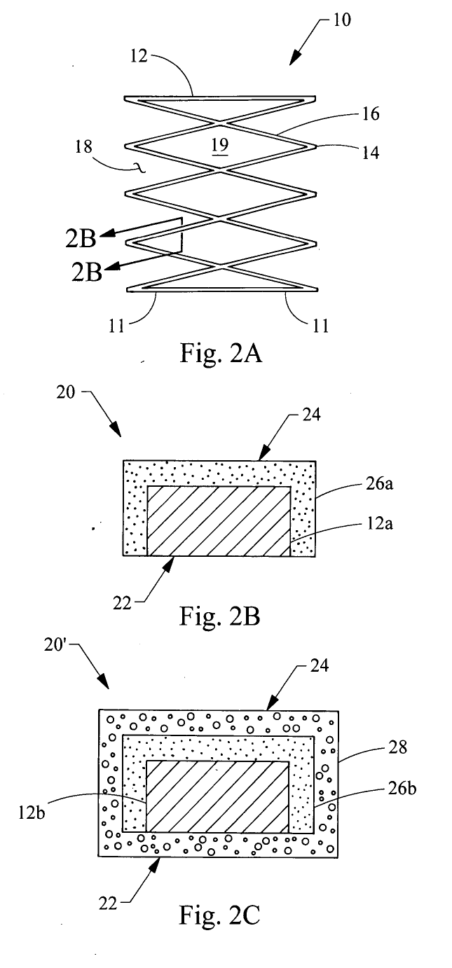

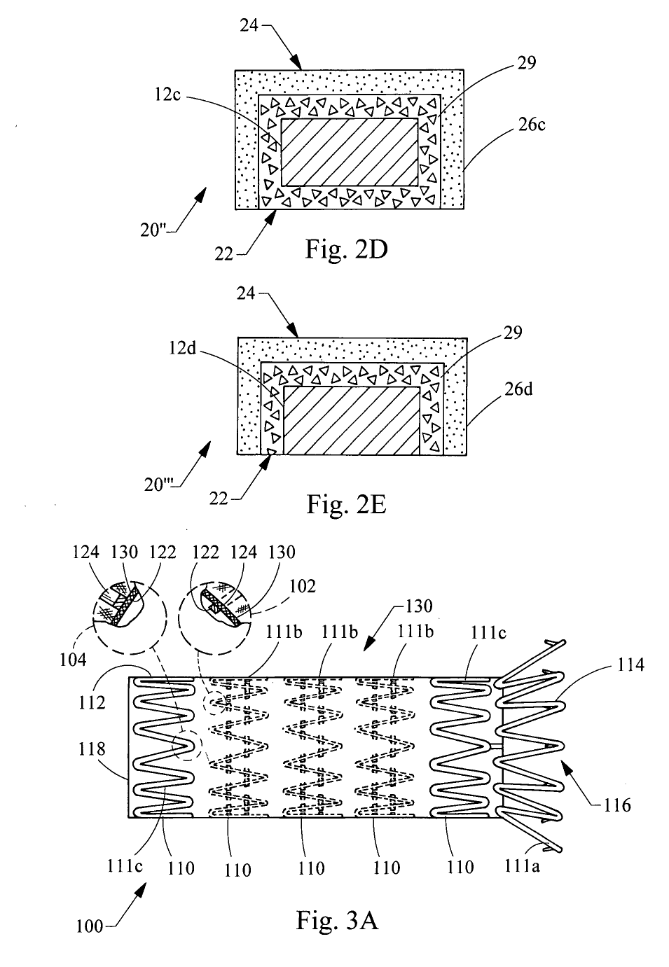

In one aspect, the medical device may be a stent 10. The stent may have any configuration adapted to maintain the lumen of a body vessel at a desired degree of patency. FIG. 2A shows a side view of a stent 10 configured as a radially-expandable frame 12 formed from a plurality of interconnected struts 16 and bends 14 forming a pair of longitudinally joined hoop members 11. Alternatively, a stent may include one or a plurality of radially-expanding stents such as Z-STENTS®, which are available from Cook, Incorporated (Bloomington, Ind.). The frame 12 defines a tubular lumen 18 and defines a plurality of openings 19 between the lumen 18 and the exterior surface of the frame.

The frame 12 can be formed from any suitable biocompatible material providing properties suited for an intended application, such as desired rigidity or flexibility. Stent 10 is capable of providing circumferential support while, at the same time, being axially flexible. The stent frame 12 may be formed by forming the desired pattern directly out of a tube, e.g. by laser cutting or chemical etching. Alternatively, the desired pattern may be formed out of a flat sheet, e.g. by laser cutting or chemical etching, and then rolling that flat sheet into a tube and joining the edges, e.g. by welding. Any other suitable manufacturing method known in the art may be employed for manufacturing a stent in accordance with the invention. Furthermore, stents may be formed by etching a pattern into a material or mold and depositing stent material in the pattern, such as by chemical vapor deposition or the like. Such stents may be formed of plastic, metal or other materials and may exhibit a multitude of configurations. The metals from which such stents are formed may include stainless steels, titanium, Nitinol, and tantalum among others.

The frame 12 can be configured in any suitable pattern providing desired hoop strength and flexibility within a body vessel. The stent 10 may be moveable from a radially compressed state to the radially expanded state shown in FIG. 2A. In the radially compressed state, the stent 10 may be symmetrically radially compressed about the longitudinal axis within the center of the tubular lumen 18, and loaded into a suitable catheter-based endolumenal delivery system. The stent 10 can be positioned at a point of treatment within a body vessel using the delivery system, and radially expanded by any suitable means to the radially expanded deployed state shown in FIG. 2A. Means for expanding the stent 10 can include inflation of a balloon within the tubular lumen 18 of the stent, or self-expansion of the stent 10 upon removal of a means for radially constraining the stent in the radially compressed state. The frame may be configured and formed from materials that provide balloon-expandable or radially-expanding structures.

The frame 12 may be a frame configured for treatment or prevention of aortic dissections. Specifically, the frame for treatment of aortic dissection may be configured to provide a radially outward force against the surface of an aorta upon implantation, providing a therapeutically effective radial force directed against the intima so as to compress the intimal, medial and/or adventitial layers of the aorta against one another, thereby preventing or mitigating aortic dissection. A self-expanding frame may be selected to have a self-expanded radius greater than the radius of the site of implantation within the aorta. The site of the frame may be selected based on medically appropriate criteria to provide a desired amount of radial force against the intimal wall of the aorta to treat or prevent aortic dissection. The frame may be formed from a self-expanding material, such as the nickel-titanium alloy NITINOL®, and may have any suitable configuration of struts and bends. For example, the frame can configured as a stent 10 as shown in FIG. 2A. Optionally, one or more frames having the configuration of stent 10 can be joined longitudinally to form an elongated prosthesis of a desired length. The stent 10 can form a repeating unit cell of the elongated prosthesis, and multiple stent 10 unit cells may be joined end to end in a manner that imparts a desired amount of lateral and tortional flexibility to the elongated prosthesis. Alternatively, a single elongated prosthesis may be formed as a single unit, for example by laser cutting a cannula of a nickel-titanium alloy to form a self-expanding stent comprising a plurality of unit cells with the configuration of stent 10. Balloon-expandable materials, such as stainless steel or cobalt-chromium alloys, may also be used to form prosthetic stents for treatment of aortic dissection. Inflation of a PTA balloon may be used to place the prosthesis within the aorta, and inflation of the balloon may be regulated to provide a desired radial force against the wall of the aorta.

In one embodiment, the stent 10 or elongated prosthesis comprising a plurality of stent 10 unit cells may be coated with a releasable elastin-stabilizing compound in a manner that provides for the therapeutically effective release of the elastin-stabilizing compound into the intimal wall of the aorta. An elongated prosthesis may be delivered or implanted at any medically appropriate site within the aorta, including the proximal or distal segment of the aorta. The elongated prosthesis may have any suitable configuration of struts, bends, and openings. One example of an elongated prosthesis is a self-expanding nickel-titanium stent for percutaneous implantation sold under the tradename ZENITH®, commercially available from Cook, Incorporated (Bloomington, Ind.). Other examples include a Wallstent variety stent or a Gianturco-Roubin, Palmaz-Shatz, Wiktor, Strecker, Cordis, AVE Micro Stent, Igaki-Tamai, Millenium Stent, Cook-Z® Stent or Zilver Stent. Some exemplary stents are disclosed in U.S. Pat. Nos. 5,292,331; 6,090,127; 5,133,732; 4,739,762; and 5,421,955.

In one embodiment, the elastin-stabilizing compound may be contained within a reservoir incorporated with the medical device.

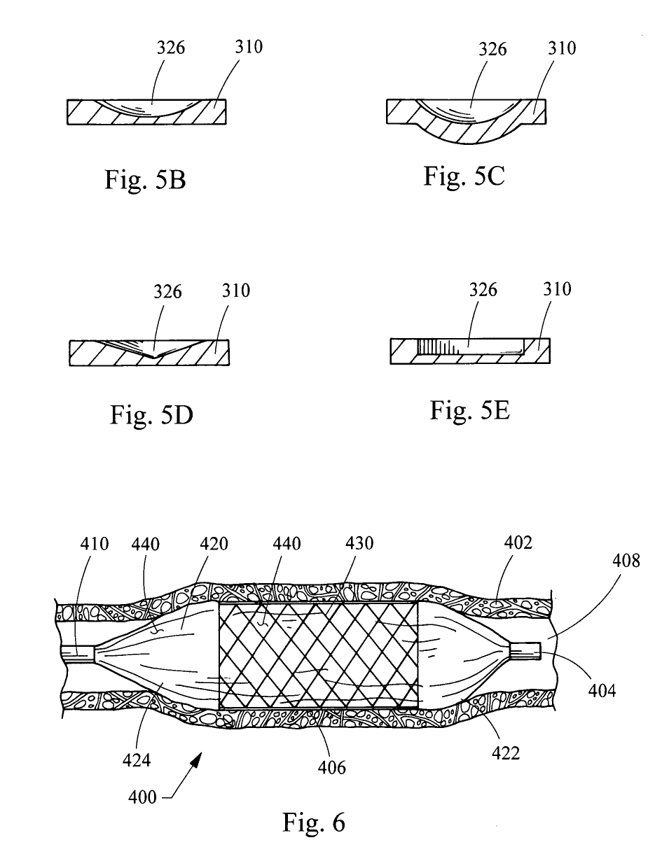

In one embodiment, the medical device may contain apertures, holes, wells, slots and the like occurring within the surface of the device for containing the elastin-stabilizing compound and optionally containing other materials, such as a biodegradable polymer, mixed with or positioned in additional layers adjoining the elastin-stabilizing compound. For example, the elastin stabilizing compound may be contained within a hole in a strut 16 or bend 14. In an alternative embodiment, the elastin-stabilizing compound may be contained within wells formed in the strut 16 and/or a bend 14 portion of the frame 12. The wells may also be configured as slots or grooves in the surface of the frame 12. Placement of the releasable elastin-stabilizing compound within a hole or well in the frame may provide the advantage of controlling the total amount of the elastin-stabilizing compound released from the medical device 10, as well as the rate of release. Referring to FIG. 5A, the ablumenal surface 324 of an arcuate frame portion 310 of a medical device frame comprises a plurality of wells 326 containing an elastin-stabilizing compound. The well 326 may contain a coating comprising the elastin-stabilizing compound alone, a mixture of the elastin-stabilizing compound with suitable polymers or a coating comprising multiple layers. FIGS. 5B-5E show cross sectional views of various well configurations along line B-B′ of frame 310. The holes or wells may have any suitable shape or size, including a concave well formed by removing a portion of the frame 310 (FIG. 5B) or formed by re-shaping a portion of the frame (FIG. 5C), a V-shape well (FIG. 5D) or a square shaped well (FIG. 5E). The holes, wells, slots, grooves and the like, described above, may be formed in the surface of the release system of the medical device 10 by any suitable technique. For example, such techniques include drilling or cutting by utilizing lasers, electron-beam machining and the like or employing photoresist procedures and etching the desired apertures.

In one embodiment, the medical device may include hollow members that are adapted to contain the elastin-stabilizing compound. Nearby, in vivo reservoirs may attach to these hollow members to supply the elastin-stabilizing compound. Medical devices and methods for delivery of therapeutic agents using hollow members adapted to contain a drug were previously described in U.S. Provisional Pat. Application Ser. No. 60/794,634 filed Apr. 25, 2006, contents of which are incorporated herein in its entirety.

The stent may be balloon expandable or radially-expanding, including elastically self-expanding and thermally self-expanding. The balloon expandable stents are typically made of a ductile material, such as stainless steel tube, which has been machined to form a pattern of openings separated by stent elements. Radial expansion can be achieved by applying a radially outwardly directed force to the lumen of a balloon expandable stent and deforming the stent beyond its elastic limit from a smaller initial diameter to an enlarged final diameter. In this process the slots deform into “diamond shapes.” Balloon expandable stents are typically radially and longitudinally rigid and have limited recoil after expansion. These stents have superior hoop strength against compressive forces but should this strength be overcome, the devices will deform and not recover. Balloon-expandable frame 12 structures may be formed from cobalt-chromium alloys or stainless steel materials. Self-expanding stents, on the other hand, may be fabricated from either spring metal or shape memory alloy wire, which has been woven, wound or formed into a stent having interstices separated with wire stent elements. When compared to balloon-expandable stents, these devices have less hoop strength but their inherent resiliency allows them to recover once a compressive force that results in deformation is removed. Other suitable frame materials include thermoformable polymers, such as polyethylene and polyurethane, and bioabsorbable polymer materials.

Several bioabsorbable, biocompatible polymers have been developed for use in medical devices, and have been approved for use by the U.S. Food and Drug Administration (FDA). These FDA-approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although some more crystalline forms can biodegrade more slowly. Biodegradable polymers that can be used to form the support frame of a medical device, or can be coated on a frame, include a wide variety of materials. Examples of such materials include polyesters, polycarbonates, polyanhydrides, poly(amino acids), polyimines, polyphosphazenes and various naturally occurring biomolecular polymers, as well as co-polymers and derivatives thereof. Certain hydrogels, which are cross-linked polymers, can also be made to be biodegradable. These include, but are not necessarily limited to, polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-alpha-hydroxy acids, trimethylene carbonate, poly-beta-hydroxy acids, polyorganophosphazines, polyanhydrides, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, modified polysaccharides and modified proteins. Some specific examples of bioabsorbable materials include poly(epsilon-caprolactone), poly(dimethyl glycolic acid), poly(hydroxy butyrate), poly(p-dioxanone), polydioxanone, PEO/PLA, poly(lactide-co-glycolide), poly(hydroxybutyrate-co-valerate), poly(glycolic acid-co-trimethylene carbonate), poly(epsilon-caprolactone-co-p-dioxanone), poly-L-glutamic acid or poly-L-lysine, polylactic acid, polylactide, polyglycolic acid, polyglycolide, poly(D,L-lactic acid), L-polylactic acid, poly(glycolic acid), polyhydroxyvalerate, cellulose, chitin, dextran, fibrin, casein, fibrinogen, starch, and hyaluronic acid.

At least a portion of the medical device frame 12 may be coated with one or more elastin-stabilizing compounds (“bioactive compound”), such as a phenolic tannin compound. The bioactive compound may be releasably associated with the frame 12 in any suitable manner that provides for the release of a therapeutically effective amount of the bioactive compound from the device upon placement of the frame 12 within a body vessel. For example, the bioactive compound may be adhered to a surface of the frame 12. Referring to FIG. 2A, the frame 12 comprises a lumenal surface defining the lumen 18 and an ablumenal surface positioned opposite the lumenal surface. FIG. 2B shows a cross section 20 view of a coated portion of the frame 12 along the line marked 2B-2B in FIG. 2A. The coating 26a comprises an elastin-stabilizing compound and can have any suitable composition or configuration that provides for a therapeutically effective release of the bioactive compound within a body vessel. For example, the coating can optionally comprise a polymer matrix, such as a biodegradable polymer or a porous biostable polymer mixed with the elastin-stabilizing compound. The coating 26a in FIG. 2B may be applied to the ablumenal surface 24 of the frame portion 12a, however the coating 26a could be applied to the lumenal surface 22 in addition to, or instead of, application to the ablumenal surface 24.

The coating may optionally comprise multiple layers. As such, these multiple layers may include varying amounts of elastin-stabilizing compound(s) creating a drug gradient. Two, three, four or more layers including the elastin stabilizing compound(s) are contemplated. For example, FIG. 2C shows an alternative cross section 20′ view of a coated portion of frame 12 along the line marked 2B-2B in FIG. 2A. The coating comprises two layers: a first layer 26b comprising an elastin-stabilizing compound positioned on the ablumenal side 24 of frame portion 12b, and a second layer 28 positioned over the lumenal side 22 and the ablumenal side 24 of the first layer 26b. The second layer 28 can provide for a slower rate of release of the elastin-stabilizing compound, for example by providing a porous diffusion barrier. The second layer 28 can comprise a biodegradable elastomer, such as poly(lactic acid), or a porous biostable material, such as parylene or a poly(alkyl)methacrylate (e.g., poly(butyl)methacrylate). FIG. 2D shows an alternative cross section 20″ view of a coated portion of frame 12 along the line marked 2B-2B in FIG. 2A. The coating comprises two layers: a first layer 29 positioned over the lumenal side 22 and the ablumenal side 24 of frame portion 12c, and a second layer 26c positioned over the ablumenal side 24 of the first layer. The second layer 26c comprises an elastin-stabilizing compound, and optionally comprises other materials such as biodegradable or biostable polymer matrix-forming components. The first layer 29 can provide for a slower rate of release of the elastin-stabilizing compound from the second layer 26c, for example by exerting an attractive force toward the second layer 26c (e.g., electrostatic or van der Waals forces). The second layer 28 can comprise a biodegradable elastomer, such as poly(lactic acid), or a porous biostable material, such as parylene or a poly(alkyl)methacrylate (e.g., poly(butyl)methacrylate). In other configurations, elastin-stabilizing compounds may be linked to the surface of the drug release system without the need for a coating by means of detachable bonds and release with time. In yet other configurations, elastin-stabilizing compounds may be included as a separate layer (separate carrier layer that includes elastin-stabilizing compound(s)) that may be attached or placed near the frame 12. These bioactive compounds may be removed by active mechanical or chemical processes, or may be in a permanently immobilized form that presents the compounds at the implantation site. Multiple layers of bioactive compounds, or mixtures of carrier material/bioactive compounds, separated by polymer layers may be present to form a multilayer coating on a medical device. As discussed above, these layers may include varying amounts of the elastin-stabilizing compound(s). In certain embodiments, different bioactive compounds may be present in the different layers. For example, different phenolic tannins, such as PGG and tannic acid, may be present in different layers. In another embodiment, bioactive agents different from phenolic tannins may also be included in addition to the phenolic tannins in the same layers or different layers. Examples of other suitable bioactive agents are described below.

In one embodiment, the coating may also be confined to the ablumenal surface. Referring to FIG. 2E, an alternative cross section 20 view of a coated portion of frame 12 along the line marked 2B-2B in FIG. 2A comprises a two-layer coating: a first layer 29 positioned over the ablumenal side 24 of frame portion 12d, and a second layer 26d positioned over the first layer. The second layer 26d comprises an elastin-stabilizing compound, and optionally comprises other materials such as biodegradable or biostable polymer matrix-forming components. The coating does not cover the lumenal side 22 of the frame.

The elastin-stabilizing compound(s) may be incorporated into the medical device in any suitable manner. The term “incorporated” means that phenolic tannins are coated, adsorbed, placed, deposited, attached, impregnated, mixed, or otherwise incorporated into the device and the layers described herein by methods known in the art. Coating layers may be applied in sequential fashion by placing the layers near the medical device and/or spraying a solution comprising a volatile solvent and a phenolic tannin to the surface of the medical device. A coating layer comprising a phenolic tannin compound may be adhered to the surface of the using an ultrasonic nozzle spray coating technique employing ultrasound to atomize a spray solution comprising the phenolic tannin, in suitable solvent, to provide a smooth and uniform coating. Optionally, the spray solution can further comprise a soluble polymer, such as a biodegradable polymer. In general, high frequency nozzles are smaller, create smaller drops, and consequently have smaller maximum flow capacity than nozzles that operate at lower frequencies. The ultrasonic nozzle can be operated at any suitable frequency, including 24 kHz, 35 kHz, 48 kHz, 60 kHz, 120 kHz or higher. A frequency of 60-120 kHz or higher may be used to atomize the solution comprising the phenolic tannin. The nozzle power can be set at any suitable level, but may be about 0.9-1.2 W or alternatively about 1.0-1.1 W. The maximum flow rate and median drop diameter corresponding to particular nozzle designs can be selected as design parameters by one skilled in the art. In one embodiment, the flow rate may be between about 0.01-2.00 mL/min.

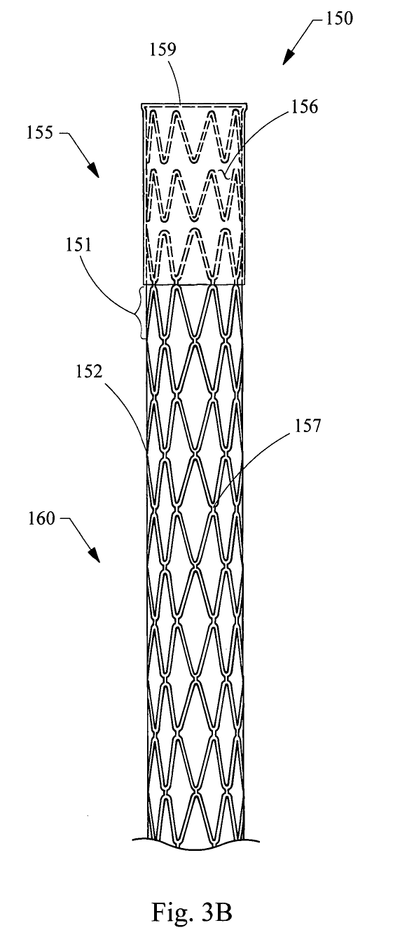

In another embodiment, the medical device may be a stent graft 100, as shown in FIG. 3A. The stent graft 100 may be formed from a graft material 130 and a frame 112. The frame 112 may comprise a plurality of longitudinally aligned hoops 110 attached to the tubular graft material 130 so as to define a cylindrical lumen 118. A distal frame hoop 111a may be flared radially outward to secure the stent graft 100 within a body vessel upon implantation. A first group of three frame hoops 111b may be positioned on the lumenal side 122 of the graft material 130 between a second group of frame hoops 111c positioned on the ablumenal 124 side of the graft material 130. A cross sectional view of a portion of a first frame hoop 111b is shown in detail view 102; a cross sectional a cross sectional view of a portion of a second frame hoop 111c is shown in detail view 104. The stent graft 100 may be moveable from a radially compressed state to the radially expanded state shown in FIG. 3A, for example by expansion of a balloon within the lumen 118, or by self expansion of the frame 112 within a body vessel. Medical devices may be packable in a compressed state within an endovascular delivery system having an outer diameter of from about 0.06 inches (5 French) to about 0.27 inches (20 French); preferably from about 0.10 inches (8 French) to about 0.22 inches (17 French); and most preferably from about 0.13 inches (10 French) to about 0.19 inches (14 French).

Resiliently compressible, self-expanding frames 112, such as self-expanding stent materials discussed above, are preferred in order to seal with the body lumen. PCT Application WO 98/53761, hereby incorporated by reference in its entirety, discloses a number of details concerning stents, stent grafts, and a method for implanting stent grafts into the human body.

The graft material 130 may be any suitable material for an intended use. The graft material 130 may be a woven or non-woven fabric, such as Dacron®, or may be a polymeric material such as expanded polytetrafluoroethylene (ePTFE), or may be a reconstituted or naturally derived collagenous material, such as mall intestine submucosa (SIS). Other materials suitable for use as the graft material are also contemplated.

The graft material 130 may be selected and adapted to retain a therapeutically effective amount of an elastin-stabilizing compound. The graft material may be selected from the group consisting of polyester, polyurethane (THORALON (THORATEC, Pleasanton, Calif.)), polyethylene, polypropylene and polytetrafluoroethylene. The graft material may alternatively be made from a reconstituted or naturally-derived collagenous material. Suitable bioremodelable materials may be provided by collagenous extracellular matrix materials (ECMs) possessing biotropic properties, including in certain forms angiogenic collagenous ECMs. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa.

As prepared, the submucosa material and any other ECM may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM may include other biological materials such as heparin, heparin sulfate, hyaluronic acid, fibronectin and the like.

Submucosa or other ECM materials may be derived from any suitable organ or other tissue source that usually contains connective tissues. The ECM materials processed for use as the graft material will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example at least about 1%, at least about 3%, and at least about 5% by weight in various embodiments of the invention.

The submucosa or other ECM material used as the graft material may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Submucosa or other ECM tissue used in the invention is preferably highly purified, for example, as described in U.S. Pat. No. 6,206,931. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used as the graft material in the present invention. Other collagen sources may be used in order to provide a desired amount of various collagen types including type I, III, IV and VI (Murata, et al., Atherosclerosis, 1986 June; 60(3):251-62).

One type of submucosa for use as a graft material in this invention may be derived from the intestines. Another type of submucosa for use as a graft material in this invention may be derived from a small intestine, of a warm blooded vertebrate; i.e., SIS. SIS is commercially available from Cook Biotech, West Lafayette, Ind.

The graft material 130 may be attached to the frame 112 by any suitable method, including suturing, cross linking of the graft material 130 to the frame 112, or the application of adhesive compositions to join the frame 112 to the graft material 130 or by heat or by ultrasonic bonding.

Any portion of the stent graft may be coated with or include the elastin-stabilizing compound.

In one embodiment, an elastin-stabilizing compound may be coated or positioned within or on the graft material 130. One or more phenolic tannin bioactive materials may be incorporated in or coated on a graft material 130 by any suitable method. Various methods of coating, impregnating, or lining the graft fabric with the bioactive compounds may be utilized and are known in the art. For example, the bioactive compounds may be deposited onto the graft fabric by spraying, dipping, pouring, pumping, brushing, wiping, vacuum deposition, vapor deposition, plasma deposition, electrostatic deposition, epitaxial growth, or any other method known to those skilled in the art. The type of coating or vehicle utilized to immobilize the bioactive compound to the graft material may vary depending on a number of factors, including the type of the medical device, including the graft material, the type of bioactive compound, and the rate of release thereof. Bioactive compounds may be incorporated into or mixed with the graft material during the formation of the graft material. The bioactive compound may be present in a liquid, a finely divided solid, or any other appropriate physical form when the graft material solidifies from a solution. In another embodiment, bioactive compounds may be incorporated into a solid form of the graft material, for example by spraying or dipping. Optionally, the graft material, or a coating applied thereto, may include one or more additives, for example, auxiliary substances, such as diluents, carriers, excipients, stabilizers, or the like. Optionally, an adhesion-promoting coating layer may be applied to the graft material prior to coating it with the bioactive compound. The adhesion promoting layer can be configured to provide a durable coating of the bioactive adhered to the graft material. Examples of suitable adhesion promoting materials include silane and parylene polymers. The amount of bioactive compound will be dependent upon a particular bioactive employed and medical condition to be treated. In one embodiment, the bioactive compound remains on the graft material during the delivery and implantation of the medical device. Accordingly, various materials may be utilized as surface modifications to prevent the bioactive compound from coming off prematurely. These materials are known and commonly used in the art.

One particular method of coating or impregnating a graft involves impregnating the graft with the bioactive compound by applying pressure to force the compound into the interstices of the graft. Pressure or force can be applied using a number of mechanical means for impregnating a solution of the bioactive compound into the graft material. Once coated, the grafts are allowed to dry and then may be subjected to sterilizing conditions prior to introduction into the body.

In one aspect, a dry, finely subdivided bioactive compound may be blended with a wet or fluid material, such as ePTFE, used to form the graft material before the material solidifies. Alternatively, air pressure or other suitable means may be employed to disperse the bioactive compound substantially evenly within the pores of the solidified graft material 130. In situations where the bioactive compound is insoluble in the wet or fluid graft material, the bioactive compound may be finely subdivided as by grinding with a mortar and pestle or by other means. The bioactive compound may be micronized, e.g., a product wherein some or all particles are the size of about 5 microns or less. The finely subdivided bioactive compound can then be distributed desirably substantially evenly throughout the bulk of the wet or fluid ePTFE layer before cross-linking or cure solidifies the layer. Alternatively, a bioactive compound may be incorporated into the graft material 130 by mixing a crystalline, particulate material (e.g., salt or sugar) that is not soluble in a solvent into an extrudate used to make the graft material to form the extrudate; casting the extrudate solution with particulate material; and then applying a second solvent, such as water, to dissolve and remove the particulate material, thereby leaving a porous graft material 130. The graft material 130 may then be placed into a solution containing a bioactive compound in order to fill the pores. In one embodiment, the stent graft would be exposed to a vacuum during solution impregnation to insure that the bioactive compound applied to it is received into the pores. Alternatively, the bioactive compound may be coated on the outside surface of the graft material. The drug may be applied to the outside surface of the graft material such as by dipping, spraying, or painting.

Optionally, the bioactive compound may be contained within a reservoir, such as encapsulated in microparticles, such as microspheres, microfibers or microfibrils, which can then be incorporated into a graft material. Various methods are known for encapsulating bioactives within microparticles or microfibers (see Patrick B. Deasy, Microencapsulation and Related Drug Processes, Marel Dekker, Inc., New York, 1984). For example, a suitable microsphere for incorporation would have a diameter of about 10 microns or less. The microsphere could be contained within the mesh of fine fibrils connecting the matrix of nodes in the graft material. The microparticles containing the drug may be incorporated within a zone by adhesively positioning them onto the material or by mixing the microparticles with a fluid or gel and flowing them into the graft material. The fluid or gel mixed with the microparticles could, for example, be a carrier agent designed to improve the cellular uptake of the bioactive compound incorporated into the graft material. Moreover, it is well within the contemplation of the present invention that carrier agents, which may include hyaluronic acid, may be incorporated within each of the embodiments of the present invention so as to enhance cellular uptake of the bioactive compound associated with the device. The microparticles embedded in the graft material may have a polymeric wall surrounding the bioactive compound or a matrix containing the bioactive compound and optional carrier agents. Moreover, microfibers or microfibrils, which may be bioactive compound loaded by extrusion, can be adhesively layered or woven into the graft material. Alternatively, the bioactive compound may be coated on the outside surface of the graft material. The bioactive may be applied to the outside surface of the graft material by, for example, dipping, spraying, or painting.

Alternatively, the graft material may further include a coating posited over the graft material. The coating may include, for example, a biocompatible hydrophilic material, such as hydrophilic polymer. Hydrophilic polymers that may be suitable for use as a coating for the graft fabric material are readily and commercially available from, for example, Biosearch Medical Products, Sommerville, N.J.; Hydromer Inc. Branchburg, N.J.; Surmodics, Eden Prairie, Wis.; and STS Biopolymers, Inc., Henrietta, N.Y. For example, hydrophilic polymer may include, but not be limited to, polyethylene oxide, polyvinyl pyrrolidone, polyethylene glycol, carboxylmethyl cellulose, hydroxymethyl cellulose, and other suitable hydrophilic polymers, or a combination thereof.

The medical device may also be configured as an elongated stent graft for treatment of aortic dissections as described in published U.S. Publication No. 2004/0176832 A1, published on Sep. 9, 2004, which is incorporated by reference in its entirety. For example, FIG. 3B shows an elongated stent graft 150 in a radially expanded configuration. The elongated stent graft 150 comprises an elongated frame 152 and a biocompatible graft material cover 156 around a first end 159 of the elongated stent graft 150 to form a covered portion 155 and an uncovered frame portion 160. The elongated frame 152 is formed from a plurality of longitudinally connected hoop members 151 joined by flexible links 157. Each hoop member 151 is formed from a sinusoidal member comprising an interconnected array of struts and bends. The flexible links 157 enable each hoop member 151 to radially expand separately. The elongated stent graft 150 may have a total length of from 100 to 300 mm and a diameter when expanded of 22 to 45 mm. The covered portion 156 may have a length of from 50 to 150 mm and a diameter when expanded of 22 to 45 mm. The length of the elongated stent graft 150 may be selected based on various factors, including the nature of the aortic aneurysm or dissection, the length of aorta at the site of treatment, and the dimensions of the aneurysm or the rupture in the wall of the aorta. Optionally, the elongated stent graft 150 may include barbs at the first end 159 of the elongated stent graft 150. The elongated frame 152 may be in the form of a mesh and formed from a biocompatible and biodegradable mesh material to permit dissipation of the elongated frame 152 after a desired period of time within a blood vessel.

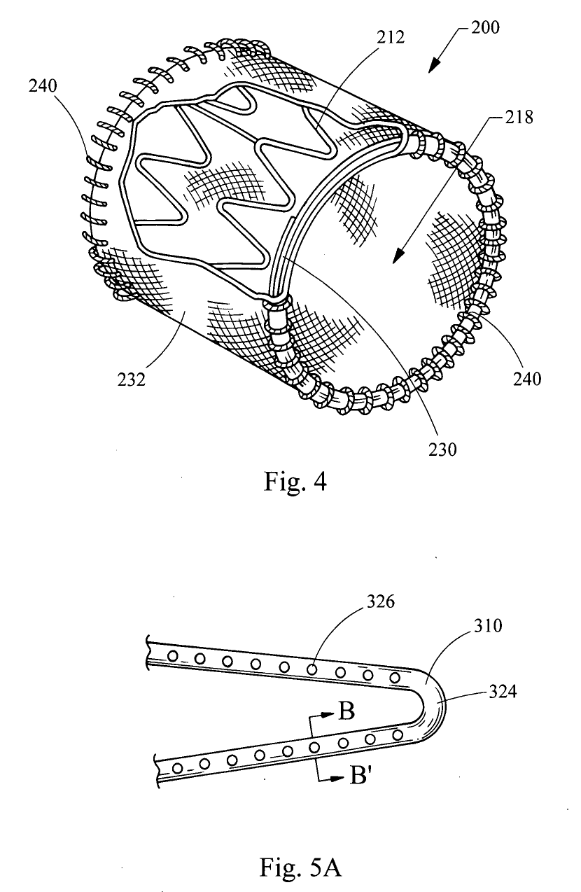

Referring to FIG. 4, a stent graft 200, may comprise a multilayered graft material construct including a frame 212 positioned between an inner tubular graft material 230 defining the lumen 218 and an outer tubular graft material 232 defining the outer surface of the stent graft 200. The frame 212 and the nested tubular graft materials 230, 232 can be joined by a plurality of sutures 240 at each end of the stent graft 200. One or more bioactive compounds can be incorporated in each of the tubular graft materials 230, 232. For example, an elastin-stabilizing agent, such as a phenolic tannin, may be included within the outer tubular graft material 232, and a second bioactive compound may be included within the inner tubular graft material 230. The second bioactive compound can be selected for retention or release into fluid flowing through the lumen 218.

A medical device may be compressible into a radially compressed delivery configuration being configured for implantation from a suitably small delivery system. In one embodiment, the delivery system has sufficient pushability, trackability and lateral flexibility. The device may be delivered to the treatment site by endovascular insertion. Preferably, the endovascular delivery system is sufficiently rigid to enable the health practitioner performing the implantation procedure to push the delivery system deep into the vascular tree of a patient, but not so rigid as to cause vascular damage during the implantation procedure. Furthermore, the delivery system would have enough lateral flexibility to allow tracking of the path of any one of the blood vessels leading to the implantation site. A delivery system, or introducer, typically comprises a cannula or a catheter, having a variety of shapes according to the intended clinical application and implantation site. The medical device may be radially collapsed and inserted into the catheter or cannula using conventional methods. In addition to the cannula or catheter, various other components may need to be provided in order to obtain a delivery system that is optimally suited for its intended purpose. These include and are not limited to various outer sheaths, pushers, stoppers, guidewires, sensors, etc. Once the device is deployed within a vessel, it expands and it can remain in place indefinitely, acting as a substitute vessel for the flow of blood or other fluids. Alternatively, if the device may be intended for temporary treatment, it can be removed after a desired period of time (hours, days, months, or years) from within the patient by conventional means.

In yet another embodiment, a medical device may be configured as a medical device delivery system comprising an elastin-stabilizing compound. The delivery system may include a structure, such as a balloon catheter, configured to deliver the medical device to a predetermined location within a body lumen of a patient and release an elastin-stabilizing compound before, during or after deployment of the medical device. Examples of balloons used for drug delivery were described in U.S. Publication No. 2004/0073190 A1, published on Apr. 15, 2004, and U.S. Publication No. 2005/0278021 A1, published on Dec. 15, 2005, the disclosures of which are incorporated by reference in their entirety.

FIG. 6 shows a portion of a distal portion of a catheter device 400 coated with the elastin-stabilizing compound. The catheter 410 may include an inflatable balloon 420 proximate to the distal end 404 of the catheter 410. Inflation of the coated balloon 420 within a body vessel 402 may place the elastin-stabilizing compound in contact with the wall 406 of the body vessel 402. The balloon 420 may be inflated to a controlled pressure (e.g., up to 1 to 20 atm) to fill the cross-section of the body lumen 408 and press the coated balloon surface 440 against the wall 406 of the body vessel lumen 408. The coated balloon surface 440 is configured to release the elastin-stabilizing compound from the surface of the balloon 420 during compression of the inflated balloon against the wall 406 of the body vessel lumen 408.

Optionally, at least a portion of the coating 440 of the expandable balloon may include the elastin-stabilizing compound mixed with, or layered with, a swellable hydrogel polymer. In one instance, the coating 440 may have a thickness in the range of about 10 to 50 microns in the swelled state. The hydrogel polymer may be selected from the group consisting of polycarboxylic acids, cellulosic polymers, gelatin, polyvinylpyrrolidone, maleic anhydride polymers, polyamides, polyvinyl alcohols, and polyethylene oxides. In general, when dry, the hydrogel coating may be on the order of about 1 to 10 microns thick. Typically, the hydrogel coating thickness may swell by about a factor of 6 to 10 or more when the hydrogel coating is hydrated. For example, a 1 to 3 microns thick hydrogel coating, when dry, may swell to about 10-30 microns thickness when hydrated. For example, a hydrogel coating on an angioplasty balloon may be coated on the surface of a balloon catheter (e.g., polyethylene) by applying a solution of 4,4′ diphenylmethane diisocyanate (MDI) in methylethylketone to the surface of the balloon. After drying in an air oven at 85 C for 30 minutes, the balloon may be dipped in a solution of poly(acrylic acid) in dimethylformamide (DMF) and tertiarybutyl alcohol. The balloon may be oven dried to remove solvent from the coating. The surface of the balloon becomes instantly lubricous upon exposure to water. The formation of the hydrogel is further described in U.S. Pat. No. 5,091,205. The elastin-stabilizing compound may be incorporated within the hydrogel polymer coating by, for example, dipping a hydrogel coated balloon in an aqueous solution of the elastin-stabilizing agent.

The medical device may be a balloon catheter configured to deliver the elastin-stabilizing compound and to deploy a second medical device, such as a radially-expandable stent crimped around the balloon portion of the catheter. For example, referring again to FIG. 6, a second medical device, such as a stent 430, can be crimped over a balloon coated with the elastin-stabilizing agent. Expansion of the coated balloon portion 440 of the catheter 410 can function to radially expand and deploy a stent 430, while simultaneously releasing the elastin-stabilizing compound onto the lumenal surface of the stent 430 and/or the wall 406 of the body vessel 402. The elastin-stabilizing compound can be coated on at least a portion of the inflatable balloon 420, for instance at the proximal region 424 and distal region 422 that extend longitudinally beyond a crimped stent 430. Inflation of the balloon 420 typically leads to inflation of the distal region 422 and proximal region 424 of the balloon 420 (also referred to as the “dogbone” inflation pattern). In one embodiment, the elastin-stabilizing compound may be coated on the distal region 422 and the proximal region 424 of the balloon 420 that are not enclosed by the stent 430. During delivery of a stent 430 by balloon inflation, the proximal region 424 and distal region 422 may radially expand before the portion of the balloon 420 enclosed by the stent 430, thereby delivering the elastin-stabilizing compound to the wall of the body vessel before the stent is fully expanded.

The medical device may be an infusion catheter comprising one or more drug delivery channels from the central lumen of the catheter to the outer surface of the catheter. For example, an elastin-stabilizing compound may be locally delivered in liquid form from the catheter near a point of treatment within an aorta. Optionally, the infusion catheter medical device may include one or more balloons. In one aspect, the infusion catheter includes a pair of balloons spaced longitudinally along the catheter, and one or more channels in communication with the outside surface of the catheter between the balloons. The balloons may be inflated prior to or during delivery of the elastin-stabilizing compound, localizing the elastin-stabilizing compound within an isolated segment of the body vessel between the two balloons.

The infusion catheter may also include a balloon segment with one or more pores permitting delivery of the elastin-stabilizing compound across the balloon membrane. The balloon may be inflated with air or with a solution of the elastin-stabilizing compound that is released through the balloon pores at a desired rate. The size of the pores, the viscosity and concentration of the solution comprising the elastin-stabilizing agent, as well as the inflation pressure of the balloon, may be selected to provide a desired rate of delivery of the elastin-stabilizing compound to a vessel wall upon inflation of the balloon.

A catheter may also be utilized to deliver a plurality of delivery capsules, including an elastin-stabilizing compound, which may be initially disposed over an exterior surface of an inflatable balloon. By inflating the balloon, the elastin-stabilizing compound capsules may become implanted into the interior wall of the aneurysm. Catheter may then be removed, leaving the capsules in place. The capsules may be any of a variety of conventional controlled drug delivery structures intended to release the desired drug into the aneurysmal wall or dissected aortic wall over time at a controlled rate. Optionally, the capsules may comprise hooks or other similar anchors for holding the capsules in the wall.

The elastin-stabilizing compound may also be placed on the balloon in a form of microencapsulated spheres, which may be disposed on the exterior of or extruded within the wall of a balloon associated with a balloon catheter. The balloon catheter and balloon are conventional and well known in the art. The microcapsules may be fabricated in accordance with any of the known methods for preparing microcapsules. See U.S. Pat. Nos. 4,897,268; 4,675,189; 4,542,025; 4,530,840; 4,389,330; 4,622,244; 4,464,317; and 4,943,449, the disclosures of which are incorporated herein by reference. The microcapsulated spheres may be configured to release the elastin-stabilizing compound when the balloon is inflated. As the balloon inflates, microcapsulated spheres containing the elastin-stabilizing compound can detach from the expanding balloon coating. For example, a typical dilatation catheter balloon may expand in circumference by 500% which stresses the attachment points to the microcapsulated spheres. Other examples of suitable balloons using microencapsulated spheres were previously described in U.S. Pat. No. 6,129,705, disclosure of which is incorporated by reference herein in its entirety.

In one embodiment, a photodynamic therapy (PDT) balloon catheter may be used when an elastin-stabilizing compound is formulated to be taken up at the treatment site (e.g., bond with the elastin or other constituents of the wall), then infrared, UV or visible light (of wavelength of 200 nm up to 1200 nm) may be used to activate the drug. PDT balloon catheters were previously described in U.S. Pat. Nos. 5,797,868; 5,709,653; and 5,728,068, disclosures of which are incorporated by reference herein in their entirety. Two methods for photodynamic therapy (PDT) treatment of blood vessels including use of a balloon are disclosed in the Narciso, Jr. U.S. Pat. Nos. 5,169,395 and 5,298,018, which are also incorporated by reference herein in their entirety. The elastin-based biomaterials that may be used to for photodynamic therapy were described in U.S. Pat. No. 6,372,228, which is incorporated herein by reference in its entirety.

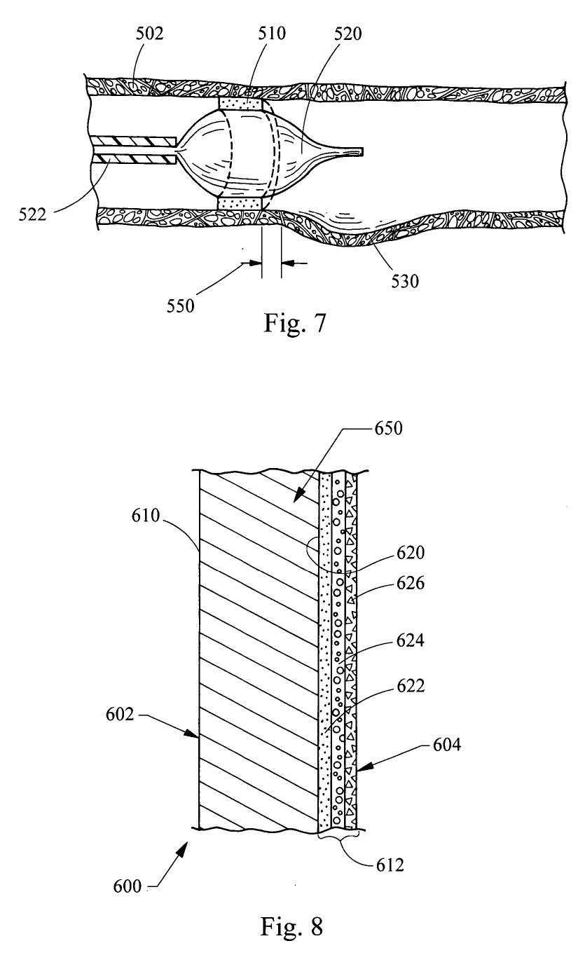

In yet another embodiment, the medical device may be configured as a flexible graft material comprising an elastin-stabilizing compound. The flexible graft material may have any suitable configuration, including a patch, sheet, tube, etc. Some specific examples include a tubular vascular graft, a flow-modifying device or an occluding device adapted for implantation within a body vessel or aneurysmal sac. The flexible graft material may be formed from any suitable material, including those described above with reference to the graft material for use with a stent graft. Exemplary materials include polyester, polyurethane, polyethylene, polypropylene, polytetrafluoroethylene (including ePTFE), reconstituted or naturally-derived collagenous material (e.g., ECM materials possessing biotropic properties, including in certain forms angiogenic collagenous ECMS). The elastin-stabilizing compound may be coated on or impregnated into a graft material in any suitable manner, including the methods for attaching the elastin-stabilizing compound to a graft material. FIG. 7 shows a flexible graft material 510 configured as a ring of an ECM material impregnated with a therapeutically-effective amount of an elastin-stabilizing compound. The flexible graft material 510 may be placed around a balloon 520 portion of a delivery catheter 522 within a body vessel 502 comprising an aneurysm 530. The flexible graft material 510 may be delivered via delivery catheter 522 placing the flexible graft material 510 around the balloon 520, placing the balloon 520 at a desired implantation site within a body vessel lumen, and expanding the balloon 520 within the body vessel to bring the flexible graft material 510 into contact with the wall of the body vessel 502 in a manner that permits adhesion of the flexible graft material 510 to the body vessel 502. Optionally, the balloon 520 may be coated with an elastin-stabilizing compound in addition to, or instead of, providing a flexible graft material 510 comprising an elastin-stabilizing compound. The site of implantation may be positioned at a therapeutically effective distance 550 from an aneurysm 530. The ablumenal surface of the flexible material 510 can be configured to permit adhesive contact with the internal wall of a body vessel. For example, the ablumenal surface of the flexible graft material 510 may have a corrugated or porous morphology or may include an adhesive substance. In one embodiment, the ablumenal surface of the flexible graft material 510 includes a desired amount of elastin-stabilizing compound releasably attached to the surface.

In one embodiment, an elastin-stabilizing compound-loaded film may be pre-mounted upon a deflated balloon catheter. The balloon catheter may be maneuvered into the desired arterial or venous location using standard techniques. The balloon may then be inflated, compressing the stent (film material) against the vessel wall and then the balloon may be deflated and removed leaving the elastin-stabilizing compound-loaded film in place. A protective sleeve (e.g., of plastic) may be used to protect the stent during its passage to the vessel and then withdrawn once the film is in the desired location.

In one embodiment, methods are provided for treating endovascular disease, such as aneurysm, and more specifically, an abdominal aortic aneurysm. The methods comprise delivering a medical device and a phenolic tannin compound to a point of treatment in a patient having an aneurysm. The phenolic tannin compound may be releasably incorporated into the medical device.

In another embodiment, the methods are provided for preventing or treating an aortic dissection. The methods comprise delivering a medical device and a phenolic tannin compound to a point of treatment in a patient having the aortic dissection, or presenting symptoms thereof. The phenolic tannin compound may be releasably incorporated into the medical device.

In some embodiments, the elastin-stabilizing compound may be releasably coated on one or more surfaces of the medical device. For example, FIG. 8 is a radial cross section 600 of a medical device formed from a medical device material 610 having a lumenal surface 610 facing the lumenal side 602 of the medical device and an ablumenal surface 620 facing toward the ablumenal side 604 of the medical device. The medical device material 610 represents a portion of any implantable medical device, including a stent frame, a graft material, a balloon portion or a catheter portion.

The medical device may include one or more coating or other layers that include elastin-stabilizing compounds. For example, the medical device shown in FIG. 8 includes a three-layer coating positioned on the ablumenal surface 620, a first coating layer 622, a second coating layer 624, and a third coating layer 626. However, coatings may have any suitable number of layers, including 1, 2, 3, 4, 5, and 6-layer coatings applied to the lumenal surface 610 and/or the ablumenal surface 620. At least one or more separate sheet layers that include elastin-stabilizing compounds embedded or otherwise included in the carrier material, which may be placed near the medical device or between the elements of the device, are also contemplated.

In one aspect, the coating 612 may form a concentration gradient of an elastin-stabilizing compound. For example, in FIG. 8, the coating 612 comprises a first coating layer 622 having a first concentration of the elastin-stabilizing compound in a carrier material, a second coating layer 624 having a second concentration of the elastin-stabilizing compound in a carrier material and a third coating layer 626 having a third concentration of the elastin-stabilizing compound in a carrier material. The carrier material may include, for example, a bioabsorbable polymer and/or a porous biostable polymer. Alternatively, the one or more coating layers may be positioned on the lumenal surface 610 instead, or in addition to, positioning coating layers on the ablumenal surface 620. The layers may optionally include the elastin-stabilizing agent in combination with other bioactive agents, and/or carrier compositions.

In another aspect, the coating 612 may include one or more layers having different compositions. For example, the first coating layer 622 may be an adhesion-promoting layer comprising a material such as parylene or silane that promotes the adhesion of the second coating layer 624 to the coated medical device surface (e.g., the lumenal surface 610 or the ablumenal surface 620). The second coating layer 624 may include the elastin-stabilizing compound and optionally comprise a carrier material such as a bioabsorbable polymer. The third coating layer 626 may include a porous material through which the elastin-stabilizing compound in the second coating layer 624 may diffuse. Optionally, the third coating layer 626 may include a soluble material impregnated within an insoluble porous material, such that dissolution of the soluble material upon implantation of the medical device results in the formation of pores in the third coating layer 626. This or other layers may also contain adhesive material(s) that cause the layer(s) to adhere to the aorta wall.

Illustrative embodiments of the present invention have been described in considerable detail for the purpose of disclosing a practical, operative structure whereby the invention may be practiced advantageously. The designs described herein are intended to be exemplary only. The invention encompasses embodiments both comprising and consisting of the elements described herein with reference to the illustrative embodiments.

Combination Therapy

In one embodiment, the invention provides a medical device comprising one or more elastin-stabilizing compounds and one or more other bioactive agents. In one embodiment, therapeutically effective amounts of the elastin-stabilizing compound and bioactive agents are provided. Examples of suitable elastin-stabilizing compounds were described above.

Other bioactive agents may be incorporated with the medical device using the methods which were described above in connection with incorporating the elastin-stabilizing compounds with the medical device of this invention.

Other bioactive agents that may be incorporated with the medical device of this invention include MMPs inhibitors, including endogenous inhibitors, such as tissue inhibitors of MMPs (TIMPs) and α-macroglobulins, and synthetic inhibitors, such as chelating agents (e.g., EDTA and 1,10-phenanthroline), peptides, antibodies, and the like. Agents that would enhance function of TIMPs may also be used.

Any suitable tetracycline, including tetracycline per se, or tetracycline-derivative compounds, such as for example, doxycycline hydrate, doxycycline aureomycin and chloromycin may be included. Preferred tetracycline compounds include CMTs (CMT that lack the dimethylamino group at position 4 of the ring structure of tetracycline, including 4-dedimethylaminotetracycline (CMT-1), 4-dedimethylamino-5-oxytetracycline, 4-dedimethylamino-7-chlorotetracycline (CMT-4), 4-hydroxy-4-dedimethylaminotetracycline (CMT-6), 5a,6-anhydro-4-hydroxy-4-dedimethylaminotetracycline, 6-demethyl-6-deoxy-4-dedimethylaminotetracycline (CMT-3; COL-3), 4-dedimethylamino-12a-deoxytetracycline (CMT-7), and 6-α-deoxy-5-hydroxy-4-dedimethylaminotetracycline (CMT-8); tetracyclines modified at the 2 carbon position to produce a nitrile, e.g., tetracyclinonitrile; 6-α-benzylthiomethylenetetracycline, the mono-N-alkylated amide of tetracycline, 6-fluoro-6-demethyltetracycline, and 11α-chlorotetracycline).

In another embodiment beta blockers may be included. Beta blockers include acebutolol, atenolol, betaxolol, bisoprolol, carteolol, carvedilol, esmolol, labetolol, metoprolol, nadolol, penbutolol, pindolol, propranolol, and timolol.

Other bioactive agents useful in embodiments of this invention include cyclooxygenase-2 (COX-2) inhibitors; angiotensin-converting enzyme (ACE) inhibitors; glucocorticoids; nitric acid synthase (NOS) inhibitors; other anti-inflammatories; anti-oxidants; and cellular adhesion molecules (CAMs).

COX-2 inhibitors include Celecoxib, Rofecoxib, Valdecoxib, Etoricoxib, Parecoxib, all of which are available in pharmacological preparations. Additionally, COX-2 inhibition has been demonstrated from herbs, such as green tea, ginger, turmeric, chamomile, Chinese gold-thread, barberry, baikal skullcap, Japanese knotweed, rosemary, hops, feverfew, and oregano; and other agents, such as piroxican, mefenamic acid, meloxican, nimesulide, diclofenac, MF-tricyclide, raldecoxide, nambumetone, naproxen, herbimycin-A, and diaryl hydroxyfuranones.

NSAIDs that may be used in embodiments according to the present invention include ketoralac tromethamine (Toradol), indomethacin, ketorolac, ibuprofen and aspirin among others. Additionally, steroidal based anti-inflammatories, such as methylprednisolone, dexamethasone or sulfasalazine may be provided. Other suitable anti-inflammatory agents include cyclosporine A and azathioprine.

Another type of suitable bioactive agents are anti-oxidants, such as curcumin, vitamins, and vitamin constituents, such as α-tocopherol and β-carotene.

Yet other bioactive agents include ACE inhibitors, such as captopril, enalapril, losartan and lisinopril and the active forms of several ACE inhibitor prodrugs on the market.

Another group of bioactive agents that may be used include cathepsin inhibitors. Cathepsin inhibitors may be classified as cysteine proteinase inhibitors, aspartic proteinase inhibitors, or serine proteinase inhibitors. For a comprehensive review of cathepsin inhibitors see Kim W. and Kang K, “Recent developments of cathepsin inhibitors and their selectivity,” Expert Opin. Ther. Patents (2002) 12(3), pp 419-432. The medical devices comprising cathepsin inhibitors were previously described in U.S. Provisional Application No. 60/755,961, filed Jan. 3, 2006, which is incorporated herein by reference in its entirety

Other bioactive agents, such as the NOS inhibitors, including aminoguanidine are also useful in combination with the elastin stabilizing compounds of the present invention.

The invention also provides medical device coatings comprising the elastin stabilizing compounds in combination with one or more bioactive agents described in U.S. Pat. No. 5,834,449; U.S. Publication Nos. 2005/0266043 A1, published on Dec. 1, 2005, and 2006/0004441 A1, published on Jan. 5, 2006, which are incorporated herein by reference.

In addition to the embodiments described above, the invention includes combinations of the preferred embodiments discussed above, and variations of all embodiments.

We claim:

1. A medical device and an elastin-stabilizing compound, the medical device being adapted to release the elastin-stabilizing compound within a body lumen of a patient.

2. The device of claim 1, wherein the medical device is implantable.

3. The device of claim 1, wherein the elastin-stabilizing compound is a phenolic tannin.

4. The device of claim 3, wherein the phenolic tannin is a hydrolyzable tannin or a condensed tannin.

5. The device of claim 3, wherein the phenolic tannin is selected from the group consisting of gallotannins, ellagitannins, epicatechins, catechins, proanthocyanidins, derivatives, and mixtures thereof.

6. The device of claim 3, wherein the phenolic tannin is selected from the group consisting of compounds: tannic acid; β-1,2,3,4,6-Pentagalloyl-O-D-Glucopyranose; β-1,2,2,3,6-Pentagalloyl-O-D-glucose; 1,2,3,6-tetragalloyl glucose; 1,3,4,6-tetragalloyl glucose; Aceritannin=2,6-di-O-galloyl-1,5-anhydro-D-glucitol; Hamamelitannin; Ellagitannin; Eugeniin; Casuarictin; Corilagin; Geraniin; Davidiin; Castalagin; Vescalagin; Euphorbin; Oenethein B; Epicatechenin; Catechin; Epigallocatechin; Gallocatechin; Epiafzelechin; Afzelechin; Epicatechin (4β->8)-catechin; Epicatechin (4β->8)-epicatechin; Catechin (4α->8)-catechin; Catechin (4α->8)-epicatechin; Sorghum procyanidin; epicatechin-[(4b->8)-epicatechin]15-(4b->8)-catechin; Aurantinidin; Cyanidin; Delphinidin; Europinidin; Luteolinidin; Pelargonidin; Malvidin; Peonidin; Petunidin; Apigeninidin; Robinetinidin; Fisetinidin; Guibourtinidin; Robinetinidol- (4α->8)-catechin-(6α->4)-robinetinidol; Profisetinidin; Epicatechin (2β-->7,4β-->8)-Lepicatechin; Leucofisetinidin; Leucopelargonidin; Leucocyanidin; Leucodelphinidin; Leucoapigeninidin; Leucoluteolinidin, derivatives, and mixtures thereof.

7. The device of claim 3, wherein the phenolic tannin is pentagalloyl glucose (PGG).

8. The device of claim 1, wherein the medical device is a stent, the elastin-stabilizing compound releasably associated with the stent.

9. The device of claim 8, wherein the stent comprises a plurality on interconnected struts and bends and the elastin-stabilizing compound is releasably associated with the struts, bends, or a combination thereof.

10. The device of claim 8, wherein the stent comprises a plurality of Z-stents.

11. The device of claim 8, further comprising a coating comprising the elastin-stabilizing compound.

12. The device of claim 11, wherein the coating comprises one or more layers containing the elastin stabilizing compound and a bioabsorbable polymer.

13. The device of claim 1, wherein the medical device is a stent graft comprising a support frame attached to a flexible tubular covering, the elastin-stabilizing compound releasably associated with at least a portion of the stent graft.

14. The device of claim 1, wherein the medical device comprises at least one surface adapted for contact with a body vessel wall and the elastin-stabilizing compound coated on at least a portion of the at least one surface.

15. The medical device of claim 1, further comprising an elongated member having a lumen extending longitudinally along the length of the elongated member, the elongated member having an ablumenal surface and a lumenal surface, wherein the elastin-stabilizing compound is releasably associated with at least one surface of the elongated member.

16. The device of claim 15, wherein the medical device is a stent graft having an elongated member comprising a flexible tubular covering forming at least a portion of the ablumenal surface, and a radially expandable support frame comprising a plurality of hoops attached to the elongated member, the lumen forming a fluid conduit defined by the lumenal surface, wherein the elastin-stabilizing compound is releasably associated with the ablumenal surface of the elongated member.

17. The device of claim 16, wherein the flexible tubular covering comprises ePTFE or PTFE and the support frame comprises a plurality of radially-expandable members each comprising a plurality of interconnecting struts and bends.

18. The device of claim 14 or 16, wherein the flexible tubular covering comprises a covering selected from the group consisting of polyester, polyurethane, polyethylene, polyethylene terephthalate, polypropylene, polytetrafluoroethylene, reconstituted or naturally-derived collagenous material, and small intestine submucosa.

19. The device of claim 1, wherein the medical device is a balloon catheter having an expandable surface, and a coating on the expandable surface, the coating comprising an elastin-stabilizing compound.

20. The device of claim 1, wherein the medical device is a graft comprising an elastin-stabilizing compound.

21. The device of claim 1, wherein the elastin-stabilizing compound is contained within a reservoir.

22. The device of claim 1, wherein the elastin-stabilizing compound is contained within a well or a groove.

23. The device of claim 1, wherein the elastin-stabilizing compound is in or disposed on at least one separate carrier layer.

24. The device of claim 1, further comprising a bioactive agent selected from the group consisting of matrix metalloproteinase's (Mumps) inhibitors, tetracycline, tetracycline-derivative compounds, beta blockers, cyclooxygenase-2 (COX-2) inhibitors, angiogenesis-converting enzyme (ACE) inhibitors, glucocorticoids, nitric acid synthase (NOS) inhibitors, anti-inflammatories, anti-oxidants, cellular adhesion molecules (CAMs), cathepsin inhibitors, derivatives, and mixtures thereof.

25. The device of claim 24, wherein the bioactive agent is coated over the medical device and the elastin-stabilizing compound.

26. A method for treating an aneurysm or an aortic dissection, the method comprising the step of delivering the medical device and elastin-stabilizing compound of claim 1 to a point of treatment within a subject having the aneurysm or aortic dissection.

27. The method of claim 26, wherein the elastin-stabilizing compound is pentagalloyl glucose (PGG).

28. A method of treating an aneurysm or an aortic dissection comprising radially expanding a medical device in a lumen with a balloon catheter, wherein the balloon catheter releases an elastin-stabilizing compound.

29. A method of treating an aneurysm or an aortic dissection comprising radially expanding a balloon catheter comprising an elastin-stabilizing compound in a lumen, wherein the balloon catheter releases an elastin-stabilizing compound within the lumen.

30. A kit comprising:
a medical device; and
a balloon catheter comprising an elastin-stabilizing compound.
